(19) United States
(12) Patent Application Publication
(10) Pub. No.: US 2006/0003002 A1
(43) Pub. Date: Jan. 5, 2006

(54) Pharmaceutical compositions with synchronized solubilizer release

(75) Inventors: David Fikstad, Salt Lake City, UT (US); Srinivasan Venkateshwaran, Salt Lake City, UT (US); Chandrashekar Giliyar, Salt Lake City, UT (US); Mahesh Patel, Salt Lake City, UT (US)

Correspondence Address: THORPE NORTH & WESTERN, LLP., 8180 SOUTH 700 EAST, SUITE 200, SANDY, UT, 84070, US

(73) Assignee: Lipocine, Inc.

(21) Appl. No.: 11/122,788

(22) Filed: May 4, 2005

Related U.S. Application Data
(63) Continuation of application No. 10/700,838, filed on Nov. 3, 2003.

(51) Int. Cl. A61K 31/724; A61K 31/401; A61K 31/366; A61K 9/22; A61K 31/355; A61K 31/225

(52) U.S. Cl. 424/468; 514/423; 514/460; 514/458; 514/58; 514/548

(57) ABSTRACT

Pharmaceutical compositions with synchronized solubilizer release as well as various methods associated therewith, are disclosed and described. More specifically, the aqueous solubility of a drug is enhanced by synchronized release of a solubilizer.

### PRIORITY DATA

This application is a Continuation in Part of U.S. patent application Ser. No. 10/700,838, filed on Nov. 3, 2003 which is incorporated herein by reference.

### 1. TECHNICAL FIELD

The inventions disclosed herein relate generally to pharmaceutical compositions having enhanced aqueous solubility with synchronized solubilizer release. More specifically, disclosed herein are pharmaceutical compositions of drugs such as, for example, cilostazol and carvedilol where the aqueous solubility of the drug is enhanced by synchronized release of a solubilizer.

### 2. BACKGROUND

The solubility of many therapeutic agents is a significant problem in effectively administering these drugs to patients. For example, cilostazol, an agent used to treat and prevent various cardiovascular disease, when formulated as an immediate release tablet dosage form, is absorbed following oral administration, but with minimal absolute bioavailability. Furthermore, the absorption of the immediate release tablet dosage form of cilostazol is not dose proportional, which implies solubility limited absorption. Absorption of the immediate release tablet dosage form of cilostazol, is also significantly affected by food consumption, which is another indicator of solubility limited absorption. A high fat meal significantly increases absorption of the immediate release tablet dosage form of cilostazol with Cmax increasing by about 90% and AUC by about 25%. The significant increase in cilostazol absorption caused by food consumption leads to deleterious side effects, such as headache and palpitations, when the immediate release tablet dosage form of cilostazol is administered after food consumption. Therefore, the immediate release tablet dosage form of cilostazol must be taken twice a day, at least 30 minutes before or at least two hours after breakfast.

Conventional controlled release dosage forms for drugs with solubility-limited absorption are ineffective. Without significant and sustained improvement in drug solubility, conventional controlled release of a poorly soluble drug will not improve absorption thus leading to inadequate systemic drug concentration over the desired period of time.

Accordingly, what is needed are pharmaceutical compositions and oral dosage forms for increasing the solubility of drugs, particularly of drugs with solubility limited absorption such as cilostazol. Preferably, the pharmaceutical compositions and oral dosage forms can be administered in modified release dosage forms.

### 3. SUMMARY

The present invention satisfies these and other needs by providing drug compositions having enhanced aqueous solubility with synchronized solubilizer release. More specifically, pharmaceutical compositions are provided where the aqueous solubility of the drug is enhanced by synchronized release of a solubilizer.

In one aspect, a pharmaceutical composition is provided. The pharmaceutical composition comprises a therapeutically effective amount of a drug, a solubilizer and a release modulator where the release of the drug and solubilizer are synchronized. The solubilizer significantly increases the aqueous solubility of the drug when synchronously released. Synchronized drug and solubilizer release may enable modified release and may provide modified release characteristics without compromising bioavailability. Further, synchronized drug and solubilizer may allow reduction in dose required for therapeutic effect or reduction in dose frequency. Synchronized drug and solubilizer release may also reduce side effects. Synchronized drug and solubilizer may allow administration with or without food while still maintaining an acceptable pharmacokinetic and therapeutic profile. Further, reduction in drug dosing frequency and side-effects often improves patient compliance.

In another aspect, an oral dosage form is provided. The oral dosage form comprises a therapeutically effective amount of a drug, a solubilizer and a release modulator where the release of the drug and solubilizer are synchronized. Many oral dosage forms, such as tablets, capsules, powders, etc. are specifically contemplated. As readily recognized by those of ordinary skill in the art many other dosage forms may also be used in practicing the current invention.

In still another aspect, a solid oral dosage form is provided. The oral dosage form comprises a therapeutically effective amount of a drug, a solubilizer and a release modulator where the release of the drug and solubilizer are synchronized.

### 4. BRIEF DESCRIPTION OF THE DRAWINGS

FIG. 1 illustrates aqueous solubility of cilostazol as a function of solubilizer concentration in simulated intestinal fluid without enzyme at 37° C. and pH of 6.8;

FIG. 2 illustrates cilostazol and solubilizer release from Example 6.2 [USP Apparatus 1,100 rpm, 37° C., 1000 ml simulated gastric fluid without enzyme +0.275% w/v sodium dodecyl sulfate];

FIG. 3 illustrates release of solubilizers and enhancement of cilostazol solubility from Example 6.3 [Extended release tester, 10 rpm, 37° C.; 0-2 hours: 100 ml SGF w/o enzyme, 2+ hours: 100 ml SIF s/o enzyme (pH 6.8)];

FIG. 4 illustrates release of cilostazol from Examples 6-1 and 6-2 [USP Apparatus 1,100 rpm, 37° C., 1000 ml simulated intestinal fluid without enzyme (pH 6.8)];

FIG. 5 illustrates release of carvedilol and solubilizer from Example 9-1 and 9-2 [USP Apparatus 1, 100 rpm, 37° C., 0-2 h: 1,000 ml SGF (pH 1.2); 2+ h: 1,000 ml SIF (pH 6.8)];

FIG. 6 illustrates release of carvedilol from Example 10-1 and Comparator 10-1. [Extended release tester; 10 rpm, 37° C., 100 ml SGF (pH 1.2) or 100 ml SIF (pH 20 6.8)];

FIG. 7 illustrates carvedilol plasma concentration as a function of time for Example 10-1 and Comparator 11 - 1 in a single-dose randomized crossover in healthy volunteers; 25

FIG. 8 illustrates release of zafirlukast from Examples 12-1, 12-2 and 12-3;

FIG. 9 illustrates release of zafirlukast from Examples 12-4 and 12-8; and

FIG. 10 illustrates release of pioglitazone from Examples 15-1 to 15-3.

### 5. DETAILED DESCRIPTION

### 5.1 DEFINITIONS

The singular forms “a,” “an,” and, “the” include plural referents unless the context clearly dictates otherwise. Thus, for example, reference to “the solubilizer” and “the release modulator” includes reference to one or more specific solubilizers and release modulators, reference to “an additive” includes reference to one or more of such additives, and reference to “the plasticizing agent” includes reference to one or more of such agents.

“AUC” is the area under the plasma drug concentration-versus-time curve extrapolated from zero time to infinity.

“Cma,,” is the highest drug concentration observed in plasma following an extravascular dose of drug.

“Extended period of time” refers to release over an amount of time that exceeds the time required for immediate release. Release may be extended, delayed or pulsatile.

“Drug,” “pharmaceutically active agent,” “bioactive agent,” “therapeutic agent,” and “active agent” may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

“Effective amount,” and “sufficient amount” may be used interchangeably, and refer to an amount of a substance that is sufficient to achieve an intended purpose or objective.

“Immediate release” refers to release of a drug at a rate which is not significantly modified by the method of drug formulation. The term “immediate release” or “instant release” is well known to those of ordinary skill in the art.

“Patient” includes humans. The terms “human” and “patient” are used interchangeably herein.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Prodrug” refers to a derivative of a drug molecule that requires a transformation within the body to release the active drug. Prodrugs are frequently, although not necessarily, pharmacologically inactive until converted to the parent drug. A hydroxyl containing drug may be converted to, for example, to a sulfonate, ester or carbonate prodrug, which may be hydrolyzed in vivo to provide the hydroxyl compound. An amino containing drug may be converted, for example, to a carbamate, amide, enamine, imine, N-phosphonyl, N-phosphoryl or N-sulfenyl prodrug, which may be hydrolyzed in vivo to provide the amino compound. A carboxylic acid drug may be converted to an ester (including silyl esters and thioesters), amide or hydrazide prodrug, which be hydrolyzed in vivo to provide the carboxylic acid compound. Prodrugs for drugs which have functional groups different than those listed above are well known to the skilled artisan.

“Solubilizer” refers to any substance which enhances the aqueous solubility of a drug.

“Symchronized release” refers to concurrent release of a drug and a solubilizer. Release may be extended, delayed or pulsatile.

“Treating” or “treatment” of any disease or disorder refers, in one embodiment, to ameliorating the disease or disorder (Le., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter) or both. In yet another embodiment, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### 5.2 Pharmaceutical Compositions

The present invention provides pharmaceutical compositions and oral dosage forms for increasing the solubility of drugs by synchronizing release of the drug and a solubilizer. Those of skill in the art will appreciate that other physicochemical or pharmacokinetic/pharmacodynamic problems may also be alleviated by synchronized release of drug and solubilizer. In this context, synchronized release of solubilizer and drug may be employed with a number of specific release profiles and effects, including without limitation, delayed release, extended release and pulsatile release. Moreover, as will be recognized by those of ordinary skill in the art, when an oral dosage form is used, such release profiles may effect corresponding absorption profiles.

In one embodiment, the present invention provides a pharmaceutical composition comprising a therapeutically effective amount of a drug, a solubilizer; and a release modulator where the release of the drug and solubilizer are synchronized. In one embodiment, the aqueous solubility of the drug is less than about 100 pg/ml. In another embodiment, the aqueous solubility of the drug is less than about 50 gg/ml. In still another embodiment, the aqueous solubility of the drug is less than about 25 pg/ml. Preferably, the solubilizer increases the aqueous solubility of a drug by at least about 25% in comparison to the intrinsic aqueous solubility of the drug.

In one embodiment, release is over an extended period of time. In one embodiment, the extended period of time is more than about I hour. In another embodiment, the extended period of time is more than about 2 hours. In still another embodiment, the extended period of time is between about 2 hours and about 24 hours.

In some cases synchronized release may be assessed by assay and determination of the dissolution or release rate of the drug and the solubilizer. Synchronized release is exhibited if the drug and the solubilizer are concurrently released, i. e., the amount of drug and solubilizer released as a function of time are correlated. Preferably, the correlation coefficient drug and solubilizer release is about greater than about 0.80, more preferably, greater than about 0.90, most preferably, greater than about 0.95. In one embodiment, synchronized release may be assessed by measuring drug release in a dissolution experiment in which a dosage form is exposed to a non-solubilizing dissolution media (e.g., simulated gastric fluid, simulated intestinal fluid, or water). The release of drug and solubilizer are synchronized when the release occurs over an extended period of time and the observed aqueous solubility of the drug in the dissolution media is enhanced or elevated by more than 25% relative to intrinsic solubility of the drug over the extended period of time. In another embodiment, synchronized release can be assessed by the in vivo blood level profile. The dose-normalized Cm. of a synchronized solubilizer release dosage form may be reduced relative to a non-synchronized solubilizer release control while producing a comparable or greater dose-normalized AUC.

Examples of drugs which may benefit from synchronized release of drug and solubilizer include, without limitation, acamprosate, acebutolol, acitretin, alfaxalone, amlodipine, amiodarone, amoxicillin, amprenavir, anagrelide, anastrazole, atenolol, atovaquone, atorvastatin, avasimibe, azathioprine, azithromycin, bacampicillin, beclomethasone, betaxolol, bicalutamide, bisoprolol, bosentan, bucindolol, budesonide, buproprion, carvedilol, candesartan cilexetil, carbamezepine, carbidopa, celecoxib, cetirizine, chenodeoxycholic acid, ciclesonide, cilostazol, ciprofloxacin, citalopram, clarithromycin, clobetasol, clonazepam, clopidogrel, clozapine, dehydroepiandrosterone, dehydroepiandrosterone sulfate, delaviridine mesylate, desogestrel, dihydroergotamine, dianabol, dilevalol, dipyridamole, docetaxel, donezepil, desloratadine, dutasteride, econazole, efivarenz, enlopitant, entacapone, eplerenone, eprosartan, ergotamine, esmolol, estazolam, etoprolol, etoricoxib, everolimus, exemestane, fenofibate, fexofenadine, fluconazole, fluphenazine, frovatriptan, granisetron, hydrocodone, irbesartan, isradipine, itasetron, itraconazole, labetalol, lamotrigine, lansoprazole, lercanidipine, letrozole, levadopa, levofloxacin, loratadine, lorazepam, lovastatin, mefloquin, megestrol, megestrol acetate, meloxicam, metaxolone, metolazone, mifepristone, mirtazapine, modafinil, morphine, mometasone, nadalol, nefazodone, nevibulol, nifedipine, nefinavir, nimodipine, nisoldipine, norethindrone, norethindrone acetate, norfloxacin, nortestosterone, olanzapine, olmesartan medoxomil, ondasetron, oxacarbezapine, oxaprozin, oxprenolol, paroxetine, penicillin, pergolide, phenazopyridine, pioglitazone, pimecrolimus, pitavastatin, pregnanediol, pregnanolone, pregnenolone, allopregnanolone, epiallopregnanolone, progesterone, propafenone, propanolol, quetiapine, raloxifene, ramipril, ranolazine, rifapentin, risperidone, ritanovir, rivastigmine, rofeconxib, ropinorole, rosiglitazone, rosuvastatin, salmeterol, saquinavir, sertraline, sildenafil, sirolimus, sotalol, simvastatin, sparfloxacin, spironolactone, stavudine, sulfamethoxazole, sumatriptan, tacrolimus, tadalafil, tegaserod, tamsulosin, telmisartan, terbinafine, terconazole, testosterone and testosterone esters, testosterone undecanoate, methyltestosterone, thalidoamide, tiagabine, tibolone, tizanidine, tolcapone, topiramate, torcetrapib, trandolapril, tramadol, triazolam, trimethoprim, valdecoxib, vardenafil, valsartan, valrubicin, ursodeoxycholic acid, voriconazole, zafirlukast, zalepelon, zileuton, ziprasidone, and zolpidem. Some preferred drugs are cilostazol, carvedilol, zafirlukast, amiodarone, fenofibrate, dronederone, risperdone, ziprasidone, simivastatin, pioglitazone or atorvastin.

One type of therapeutic agent which may benefit from synchronized release of drug and solubilizer include without limitation, drugs with poor or pH-dependent water solubility requiring modified release profiles for reasons of safety, convenience, regiospecific absorption or stability requirements. For example, weakly basic drugs (pKa less than about 9.0), which have high solubility at gastric pH and low solubility at intestinal pH may exhibit rapid absorption in the proximal gastrointestinal tract where the pH is low and the drug is predominantly in a water-soluble ionized form, and poor or no absorption in the distal gastrointestinal tract where the pH is higher and the drug is present as the less soluble free base. Such a solubility profile may be particularly undesirable for therapeutic active compounds which exhibit unwanted side-effects due to rapid initial absorption.

Antihypertensives (e.g., acebutolol, atenolol, betaxolol, bisoprolol, bucindolol, carvedilol, dilevalol, labetalol, esmolol, etoprolol, nadalol, nevibulol, oxprenolol, propanolol, sotalol) may be associated with acute hypotensive side-effects (dizziness, light-headedness, and syncope) due to rapid initial absorption. Accordingly, poorly water-soluble or basic antihypertensives are drugs, such as those listed above, which may benefit from benefit from synchronized release of drug and solubilizer.

Carvedilol, (1-(9H-Carbasol-4-yloxy)-3-[[2-(2-methoxyphenoxy)ethyl]amino]-2-propanol, is another example of this class of pharmaceutical agents. Carvedilol is a non-selective (3-adrenergic blocking agent with a,-blocking activity and is indicated for treatment of various conditions, including cardiovascular conditions, such as hypertension and congestive heart failure. Carvedilol is weakly basic with a pKa of about 7.6 and has an extremely low water solubility (i.e., less than about 0.001 mg/ml). Carvedilol has appreciable aqueous solubility at low pH due to formation of the water-soluble ionized form, although solubility is limited to less than about I mg/ml due to the formation of a relatively insoluble hydrochloric acid addition salt.

Due to pH dependent solubility characteristics orally administered carvedilol pharmaceutical compositions may provide significant carvedilol solubility and release in the stomach due to the low pH, thus leading to elevated or rapidly increasing plasma concentrations and hypotensive side-effects. As the formulation moves through gastrointestinal tract and the pH rises, carvedilol solubility and release becomes negligible. As a result, caravedilol is required to be administered with food to delay initial release in the stomach and to reduce the potential for hypotensive adverse effects. These characteristics make carvedilol particularly well-suited for formulation in synchronized solubilizer release compositions.

Another type of therapeutic agent which may benefit from synchronized release of drug and solubilizer are poorly water soluble, poorly absorbed compounds with short plasma half-lives requiring prolonged elevated blood levels. An example of this type of agent is testosterone.

Still other types of therapeutic agents which may benefit from synchronized solubilizer release include antiarrythmics (such as amiodarone, dronederone, propafenone), antipsychotics (such as ziprasidone, risperidone) and antiparkinsonian agents (such as dopamine agonists like carbidopa, levodopa or pergolide).

Cilostazol, a well known PDE III inhibitor, may also benefit from synchronized release of drug and solubilizer. Cilostazol has been used to treat or prevent cardiovascular conditions, including cerebral ischemia, restenosis, bradychardia, peripheral arterial disease, critical limb ischemia and intermittent claudication. Cilostazol produces favorable alterations in the lipid profile of patients with dyslipidemia, particularly in diabetic patients. Synchronized cilostazol and solubilizer release may reducle drug dosing frequency from twice a day to once a day which increases patient compliance and may also reduce side effects such as headaches and palpitations. Further, synchronized cilostazol and solubilizer release may allow for cilostazol administration with or without food consumption, without unacceptable side-effects.

The above therapeutic agents are commercially available or may be synthesized using procedures known to the skilled artisan.

The pharmaceutical compositions of the present invention include a solubilizer. Preferably, the solubilizer increases aqueous drug solubility by at least 25% over the intrinsic (without solubilizer) aqueous solubility of the drug when the dosage form is dissolved in a physiologically realistic volume of aqueous solution (between about 20 and about 500 ml). In one embodiment, the solubilizer increases aqueous drug solubility by 50% or more. In another embodiment, the solubilizer increases the aqueous solubility by 100% or more. It should be understood that mixtures of the solubilizers below are within the scope of the present invention.

A variety of suitable solubilizers may be used as long as the aqueous solubility of the drug is increased. Preferably, the solubilizers are polyoxyethylene-polyoxypropylene (POE-POP) block copolymers, cyclodextrins (e.g., (3-cyclodextrin, y-cyclodextrin), cyclodextrin derivatives (e.g., sulfobutyl or hydroxypropyl ethers), bile acids, bile acid derivatives, sterol derivatives, alcohols, particularly, fatty alcohols and fatty alcohol derivatives, acids, particularly fatty acids and fatty acid derivatives and tocol derivatives. More preferably, the solubilizers are polyoxyethylene-polyoxypropylene (POE-POP) block copolymers, cyclodextrins, cyclodextrin derivatives, fatty acid derivatives and tocol derivatives.

Preferred fatty acids and alcohols are the C6-C22 fatty acids and alcohols, such as stearyl alcohol, capric acid, caprylic acid, lauric acid, myristic acid, stearic acid, oleic acid, linoleic acid, linolenic acid, arachnidoic acid, behenic acid, and their corresponding pharmaceutically acceptable salts. Preferred fatty acid and fatty alcohol derivatives include sodium dioctyl sulfosuccinate, sodium lauryl sulfate, amide esters (e.g., lauric acid diethanolamide, sodium lauryl sarcosinate, lauroyl carnitine, palmitoyl carnitine and myristoyl carnitine), esters with hydroxy-acids (e.g., sodium stearoyl lactylate); sugar esters [e.g., lauryl lactate, glucose monocaprylate, diglucose monocaprylate, sucrose laurate, sorbitan monolaurate (Arlacel® 20), sorbitan monopalmitate (Span-40), sorbitan monooleate (Span-80), sorbitan monostearate and sorbitan tristearatej, lower alcohol fatty acid esters [e.g., ethyl oleate (Crodamol EO), isopropyl myristate (Crodamol IPM) and isopropyl palmitate (Crodamol IPP)], esters with propylene glycol [e.g., propylene glycol monolaurate (Lauroglycol FCC), propylene glycol ricinoleate (Propymuls), propylene glycol monooleate (Myverol® P-06), propylene glycol monocaprylate (Capryol® 90), propylene glycol dicaprylate/dicaprate (Captex® 200) and propylene glycol dioctanoate (Captex 800)], esters with glycerol [e.g., glyceryl monooleate (Peceol), glyceryl ricinoleate, glyceryl laurate, glyceryl dilaurate (Capmul® GDL), glyceryl dioleate (Capmul GDO), glycerol monolinoleate (Maisine®), glyceryl mono/dioleate (Capmul GMO-K), glyceryl caprylate/caprate (Capmul MCM), caprylic acid mono/diglycerides (Imwitor® 988), mono- and diacetylated monoglycerides (Myvacet® 9-45)], triglycerides [e.g., corn oil, almond oil, soybean oil, coconut oil, castor oil, hydrogenated castor oil, hydrogenated coconut oil, Pureco 100, Hydrokote AP5, Captex 300, 350, Miglyol 812, Miglyol 818 and Gelucire 33/01)], mixtures of propylene glycol esters and glycerol esters [e.g., mixture of oleic acid esters of propylene glycol and glycerol (Arlacel 186)], and polyglycerized fatty acids such as polyglyceryl oleate (Plurol® Oleique), polyglyceryl-2 dioleate (Nikko] DGDO), polyglyceryl-10 trioleate, polyglyceryl-10 laurate (Nikkol Decaglyn 1-L), polyglyceryl-10 oleate (Nikkol Decaglyn 1-O), and polyglyceryl-10 mono, dioleate (Caprol® PEG 860).

Other useful fatty acid derivatives include polyethoxylated fatty acids, (e.g., PEG-8 laurate, PEG-8 oleate, PEG-8 stearate, PEG-9 oleate, PEG- 10 laurate, PEG-10 oleate, PEG-12 laurate, PEG-12 oleate, PEG-15 oleate, PEG-20 laurate and PEG-20 oleate), PEG-fatty acid diesters (e.g., PEG-20 dilaurate, PEG-20 dioleate, PEG-20 distearate, PEG-32 dilaurate and PEG-32 dioleate), PEG-fatty acid mono- and di-ester mixtures, polyethylene glycol glycerol fatty acid esters (e.g., PEG'ylated glycerol 12 acyloxy-stearate, PEG-20 glyceryl laurate, PEG-30 glyceryl laurate, PEG-40 glyceryl laurate, PEG-20 glyceryl oleate and PEG-30 glyceryl oleate) and alcohol—oil transesterification products [e.g., polyoxyl 40 castor oil (Cremophor® RH40), polyoxyl 35 castor oil (Cremophor EL or Incrocas 35), PEG-25 trioleate (TAGAT® TO), PEG-60 corn glycerides (Crovol M70), PEG-60 almond oil (Crovol A70), PEG 40 palm kernel oil (Crovol PK70), PEG-50 castor oil (Emalex C-50), PEG-50 hydrogenated castor oil (Emalex HC-50), PEG-60 hydrogenated castor oil (Cremophor RH60), PEG-8 caprylic/capric glycerides (Labrasol®), lauroyl macrogol 32 glycerides (Gelucire® 44/14), linoleoyl macrogoglycerides (Labrafil®), stearoyl macrogol-32 glycerides (Gelucire 50/13), and PEG-6 caprylic/capric glycerides (Softigen® 767)].

Particularly preferred fatty acid derivatives are esters with glycerol, propylene glycol, sorbitol, sucrose, glucose polyethylene glycol or an alpha-hydroxy acid.

Bile acid and sterol derivatives include, but are not limited to, cholate, ursodeoxycholate, chenodeoxycholate, taurochenodeoxycholate, tauroursodeoxycholate, glycochenodeoxycholate, glycoursodeoxycholate, sterols and sterol esters or ethers such as PEG-24 cholesterol ether (Solulan® C-24).

Tocol derivatives include derivatives of substances with the tocol structure [2 methyl-2-(4,8,12-trimethyltridecyl)chroman-6-ol] or the tocotrienol structure [2 methyl-2-(4,8,12-trimethyltrideca-3,7,11 -trienyl)chroman-6-of]. In particular, the mono-, di-, trimethyl- tocols, commonly known as tocopherols and their organic acid esters such as the acetate, nicotinate, succinate, and polyethylnene glycol succinate esters are included. For example, a-tocopherol acetate, a-tocopherol nicotinate, a tocopherol succinate, a.-tocopherol polyethyleneglycol (200-8000 MW) succinate, a tocopherol polyethylene glycol 400 succinate, dl-a-tocopherol polyethyleneglycol 1000 succinate, and d-a-tocopherol polyethyleneglycol 1000 succinate (Vitamin E TPGS, Eastman Chemical Co.) are included. For the practice of this invention the mixed racemic forms (e.g. all racemic or dl-) as well as the pure enantiomers (e.g. d-, 1- or RRR-) are suitable. Preferred tocol derivative include a-tocopherol esters and a polyethoxylated a-tocopherol esters. More specific preferred tocol derivatives include a-tocopherol, a-tocopherol acetate, a-tocopherol nicotinoate, a-tocopherol succinate, a-tocopherol polyethyleneglycol succinate, a-tocopherol polyethyleneglycol (200-8000 MW) succinate, a-tocopherol polyethylene glycol 400 succinate, a-tocopherol polyethyleneglycol 1000 succinate, dl-a-tocopherol polyethyleneglycol 1000 succinate, or d-a-tocopherol polyethyleneglycol 1000 succinate.

Preferred solubilizers include polyoxyl 40 castor oil, polyoxyl 35 castor oil, PEG-8 caprylic/capric glycerides (Labrasolg), sorbitan monooleate (Span-80), sorbitan monolaurate (Span 20), PEG-20 sorbitan monopalmitate (Tween 40), PEG 20 sorbitan monostearate (Tween 60), PEG-20 sorbitan monooleate (polysorbate 80 or Tween 80), glyceryl mono/dioleate (Capmul GMO-K), glyceryl caprylate/caprate (Capmul MCM), caprylic acid mono/diglycerides (Imwitor® 988), and mono- and diacetylated monoglycerides (Myvacet® 9-45), linoleoyl monoglycerides (Labrafil 2125CS), lauroyl macrogol-32 glycerides (Gelucire® 44/14), (x-tocopherol, octocopherol acetate, (x-tocopherol succinate, (x-tocopherol polyethyleneglycol (2008000 MW) succinate, a-tocopherol polyethylene glycol 400 succinate, dl-a-tocopherol polyetbyleneglycol 1000 succinate, and d-a-tocopherol polyethyleneglycol 1000 succinate.

Particularly preferred solubilizers include polyoxyl 40 castor oil, polyoxyl 35 castor oil, sorbitan monooleate, PEG-20 sorbitan monooleate (polysorbate 80 or Tween 80), linoleoyl mononglycerides (Labrafil 2125CS), lauroyl macrogol-32 glycerides (Gelucire® 44/14) and d-oc-tocopherol polyethyleneglycol 1000 succinate.

The above solubilizers are available from commercial suppliers or may be synthesized using procedures known to those of skill in the art.

The pharmaceutical compositions of the present invention also include a release modulator that synchronizes the release of the drug and the solubilizer over an extended period of time. It should be understood that mixtures of release modulators are within the scope of the present invention.

A variety of release modulator are known to those of ordinary skill in the art. Examples of suitable release modulators include, without limitation, devices such as osmotic pumps (see, e.g., Langer, supra; Sefton, 1987, CRC Crit. Ref Biomed. Eng. 14:201; Saudeket al., N. Engl. J Med. 1989, 321, 574), slowly dissolving salts or complexes (e.g., with tannic acid) or hydrolysable esters, erodible matrices (e.g., polyamides such as albumin, collagen, poly(L-glutamic-co-y-ethyl-Lglutamate, etc., polyesters like poly (s-caprolactone), poly(lactic acid), poly(glycolic acid) and their copolymers, poly(ortho esters) and polyanhydrides), ion exchange resins (such as divinylbenzene-polystyrenesulfonate copolymer), waxes (such as microcrystalline wax), insoluble carriers such as calcium sulfate, polymeric matrices, polymeric coatings, fatty acids, fatty alcohols, fatty acid derivatives, fatty alcohol derivatives (such as fatty alcohol-derived waxes like emulsifying wax or the mixed fatty acid and fatty alcohol derivatives like cetyl esters wax, camauba wax, yellow wax, and white wax) and tocol derivatives. Preferably, the release modulator is polymeric matrices, polymeric coatings, fatty alcohols, fatty acids, fatty alcohol derivatives, fatty acid derivatives or tocol derivatives.

Specific examples of polymeric materials include, without limitation, high molecular weight polyethylene glycol, cellulosics, (e.g., ethyl cellulose, methyl cellulose, hydroxypropyl cellulose, hydroxypropyl methyl cellulose (HPMC), hydroxypropyl methyl cellulose phthalate (HPMCP), hydroxypropyl methyl cellulose succinate (HPMCS), cellulose acetate, cellulose nitrate, cellulose acetate butyrate, cellulose acetate trimellitate, carboxymethylethyl cellulose, cellulose acetate phthalate), shellac, polyethylene, polyvinylchloride, polyvinyl acetate, polyvinyl acetate phthalate (PVAP), acrylic polymers, (e.g., polyacrylic acid (Carbomer), neutral polymers of methacrylates, (e.g., Eudragit NE), methacrylate copolymers with trimethylaminoethylmethacrylate as functional group (e.g., Eudragit RS, RS 100, RL, RL 100), anionic polymers of methacrylic acids and methacrylates (e.g., Eudragit L 100, L 100-55, S 100), polyvinylpyrrolidone copolymers, (e.g., polyvinylpyrrolidonevinyl acetate copolymers (Kollidon VA 64, Kollidon SR)), gelactose mannate, high molecular weight polysaccharide gums and resins (e.g., acacia, xanthan gum, tragacanth, shellac, etc.), glycuronan polymers (e.g., alginic acid and pharmaceutically available salts). Preferred polymeric release modulators are cellulose derivatives, polyvinylpyrrolidone copolymers, acrylic polymers, shellac, polyvinyl acetate phthalate and high molecular weight polysaccharide gum.

Specific examples of fatty acids or fatty alcohols and derivatives useful as release modulators include, but are not limited to, stearyl alcohol, stearic acid, hydrogenated vegetable oil, glycerol dibehenate (Compritol® 888), glycerol distearate (Precirol®), lauroyl macrogol-32 glycerides (Gelucire® 44/14), and stearoyl macrogol-32 glycerides (Gelucire 50/13), sodium steroyl lactylate, calcium steroyl lactylate, stearic acid, sucrose distearate, sucrose palmitate, sucrose dipalmitate and waxes (e.g., the mixed fatty alcohol and fatty acid derivative waxes like cetyl esters wax, nonionic emulsifying wax, yellow wax, white wax, and camauba wax). Preferred fatty acids, fatty alcohols, or derivatives include hydrogenated vegetable oil, glycerol dibehenate, glycerol distearate, glycerol dipalmitate, glycerol palmitosearate, lauroyl macrogol-32 glyceride, stearoyl macrogol-32 glyceride, calcium steroyl lactylate, stearic acid, stearoyl alcohol, sucrose distearate, sucrose palmitate, sucrose dipalmitate, carnauba wax, yellow wax, white wax, or cetyl ester wax.

Specific examples of tocol derivatives useful as release modulators include, but are not limited to, the mono-, di-, trimethyl- tocols, commonly known as tocopherols, and the organic acid esters thereof (e.g., acetate, nicitanoate, succinate, polyethylnene glycol succinate esters, etc.). For example, a-tocopherol, a-tocopherol acetate, a-tocopherol nicotinate, a-tocopherol succinate, a-tocopherol polyethyleneglycol (200-8000 MW) succinate, a-tocopherol polyethylene glycol 400 succinate are specific compounds useful as release modulators. The mixed racemic forms (e.g. all racemic or dl-), and the pure enantiomers (e.g. d-, I- or RRR-) of tocol derivatives are all useful in practicing the current invention.

Many release modulators can additionally serve as solubilizers for the drug either in the pharmaceutical composition or in aqueous dispersions (also act as a solubilizer, as defined in the previous section). Similarly, many solubilizers can additionally serve as release modulators for the drug either in the pharmaceutical composition or in aqueous dispersions (also act as a release modulator, as defined above).

The above release modulators are available from commercial suppliers or may be synthesized using procedures known to those of skill in the art.

In addition to the above-recited solubilizers and release agents, the pharmaceutical compositions can optionally include one or more additives. Specific, non-limiting examples of additives are described below.

Suitable additives include those commonly utilized to facilitate processing steps such as agglomeration, air suspension chilling, air suspension drying, balling, coacervation, comminution, compression, pelletization, cryopelletization, extrusion, granulation, homogenization, inclusion complexation, lyophilization, nanoencapsulation. melting, mixing, molding, pan coating, solvent dehydration, sonication, spheronization, spray chilling, spray congealing, spray drying, or other processes known in the art. The additive can also be pre-coated or encapsulated. Appropriate coatings are well known in the art.

The pharmaceutical compositions of the present invention can optionally include one or more solvents, i.e., additives, to increase the solubility of the active ingredient or other composition components in the carrier, as distinct from solubilizers that increase aqueous solubility of the drug. Suitable solvents for use in the compositions of the present invention include without limitation, acids (e.g., acetic acid, propionic acid, butyric acid, lactic acid, pyruvic acid, oxalic acid, malic acid, malonic acid, succinic acid, maleic acid, fumaric acid, tartaric acid, citric acid, benzoic acid, cinnamic acid, mandelic acid, salicylic acid, etc), alcohols and polyols, (e.g., ethanol, isopropanol, butanol, benzyl alcohol, ethylene glycol, propylene glycol, butanediols and isomers thereof, glycerol, pentaerythritol, sorbitol, mannitol, dimethyl isosorbide, polyethylene glycol, polypropylene glycol, polyvinylalcohol, cellulose derivatives, etc.), ethers of polyethylene glycols having an average molecular weight of about 200 to about 6000 (e.g., tetrahydrofurfuryl alcohol PEG ether (glycofurol, available commercially from BASF under the trade name Tetraglycol) or methoxy PEG (Union Carbide)) amides, (e.g., 2-pyrrolidone, 2-piperidone, caprolactam, N alkylpyrrolidone, N-hydroxyalkylpyrrolidone, N-alkylpiperidone, N-alkylcaprolactam, dimethyl acetamide, polyvinylpyrrolidone etc.), esters (e.g., ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, valerolactone and isomers thereof, butyrolactone and isomers thereof, etc.) and other solvents known in the art, such as dimethyl acetamide, dimethyl isosorbide (Arlasolve DMI (ICI)), N-methyl pyrrolidones (Pharmasolve (ISP)), monooctanoin and diethylene glycol monoethyl ether (available from Gattefosse under the trade name Transcutol). Mixtures of solvents are also within the scope of the invention. These compounds are readily available from standard commercial sources or may be synthesized using procedures known to those of skill in the art.

Preferred solvents include acetic acid, sorbitol, mannitol, glycerol, triacetin, triethylcitrate, N-methylpyrrolidone, N-hydroxyethylpyrrolidone, polyvinyl pyrrolidone, ethanol, polyethylene glycol, propylene glycol. Particularly preferred solvents include acetic acid, sorbitol, glycerol, mannitol, glycerol, ethanol, isopropanol, triacetin, polyethylene glycol, and propylene glycol.

The amount of solvent that can be included in compositions of the present invention is not particularly limited. Of course, when such compositions are ultimately administered to a patient, the amount of a given solvent is limited to a bioacceptable amount, which is readily determined by one of skill in the art. In some circumstances, it may be advantageous to include amounts of solubilizers far in excess of bioacceptable amounts, for example, to maximize the concentration of active ingredient, with excess solvents removed prior to providing the composition to a patient using conventional techniques, such as distillation or evaporation.

Other additives conventionally used in pharmaceutical compositions can be included, and these additives are well known in the art. Such additives include, but are not limited to, anti-adherents (anti-sticking agents, glidants, flow promoters, lubricants) (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding, (e.g., matrix binders (dry starch, dry sugars), film binders (PVP, starch paste, celluloses, bentonite, sucrose)), chemical binders (e.g., polymeric cellulose derivatives, such as carboxy methyl cellulose, HPC, HPMC, etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PVP, PEG, vinyl pyrrolidone copolymers, pregelatinized starch, sorbitol, glucose, etc.), bufferants, where the acid is a pharmaceutically acceptable acid, (e.g., hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, etc) and where the base is a pharmaceutically acceptable base, (e.g., an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a pharmaceutically acceptable salt of acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, and uric acid, chelating agents (e.g., EDTA and EDTA salts), coagulants (e.g., alginates) colorants or opaquants, (e.g., titanium dioxide, food dyes, lakes, natural vegetable colorants, iron oxides, silicates, sulfates, magnesium hydroxide and aluminum hydroxide), coolants, (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, citric acid, tartaric acid, gelatin, dextran, mannitol, etc.), diluents or fillers, (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, crosslinked polyvinylpyrrolidone, sodium starch glycolate and microcrystalline cellulose), hydrogen bonding agents, (e.g., magnesium oxide), flavorants or desensitizers, (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives (e.g., ascorbic acid, boric acid, sorbic acid, benzoic acid, and salts thereof, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents), (e.g., sugars, polyvinylpyrrolidone, cellulosics, polymers and alginates).

Additives can also be materials such as proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics, (e.g., dicalcium phosphate, hydroxyapitite, tricalcium phosphate, talc and titania), polyols (e.g., mannitol, xylitol and sorbitol polyethylene glycol esters) and polymers (e.g., alginates, poly(lactide coglycolide), gelatin, crosslinked gelatin and agar-agar).

It should be appreciated that there is considerable overlap between the above listed additives in common usage, since a given additive is often classified differently by different practitioners in the field, or is commonly used for any of several different functions, or may have differing functions depending on the levels in the composition. Thus, the above-listed additives should be taken as merely exemplary, and not limiting, of the types of additives that can be included in compositions of the present invention. The amounts of such additives can be readily determined by one skilled in the art, according to the particular properties desired.

The present invention encompasses various methods for the making of such pharmaceutical compositions and dosage forms. The present invention provides a method of providing drugs with enhanced solubility by synchronized solubilizer release. Pharmaceutical compositions may be manufactured by means of conventional mixing, dissolving, granulating, dragee-making, levigating, emulsifying, encapsulating, entrapping, lyophilizing processes or other methods known to those of skill in the art. Pharmaceutical compositions may be formulated in conventional manner using one or more drug, solubilizer, release modulator and/or additive which facilitate processing of drugs disclosed herein into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995). Preferred pharmaceutical compositions are formulated for oral delivery, particularly for oral modified release administration.

Pharmaceutical compositions for oral delivery may be in the form of tablets, lozenges, aqueous or oily suspensions, granules, powders, emulsions, capsules, syrups, or elixirs, for example. Moreover, where in tablet or pill form, the compositions may be coated to delay disintegration and absorption in the gastrointestinal tract, thereby providing a delayed, sustained, or pulsatile action over an extended period of time. Selectively permeable membranes surrounding an osmotically active driving compound are also suitable for orally administered pharmaceutical compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used.

For topical administration a drug may be formulated as solutions, gels, ointments, creams, suspensions, etc. as is well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In one embodiment, drugs may be formulated in accordance with routine procedures as a pharmaceutical composition adapted for intravenous administration to human beings. Typically, drugs for intravenous administration are solutions in sterile isotonic aqueous buffer. For injection, a drug may be formulated in aqueous solutions, preferably, in physiologically compatible buffers such as Hanks' solution, Ringer's solution, or physiological saline buffer. The solution may contain formulatory agents such as suspending, stabilizing and/or dispersing agents. Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When a drug is administered by infusion, it can be dispensed, for example, with an infusion bottle containing sterile pharmaceutical grade water or saline. When a drug is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

For buccal administration, the pharmaceutical compositions may take the form of tablets, lozenges, etc. formulated in conventional manner.

A drug may also be formulated in rectal or vaginal pharmaceutical compositions such as suppositories or retention enemas, e.g., containing conventional suppository bases such as cocoa butter or other glycerides.

In addition to the formulations described previously, a drug may also be formulated as a depot preparation. Such long acting formulations may be administered by implantation (for example, subcutaneously or intramuscularly) or by intramuscular injection. Thus, for example, a drug may be formulated with suitable polymeric or hydrophobic materials (for example, as an emulsion in an acceptable oil) or ion exchange resins, or as sparingly soluble derivatives, for example, as a sparingly soluble salt.

### 5.3 Therapeutic Methods of Use

The pharmaceutical compositions described herein may be administered to a patient suffering from a disease that a therapeutic agent may be used to treat. The pharmaceutical compositions may also be administered to a patient as a preventative measure against a disease that a therapeutic agent may prevent. The therapeutic agent used in a particular pharmaceutical composition is determinative of the disease that is treated or prevented by administration of the pharmaceutical composition.

In one embodiment, pharmaceutical compositions containing amiodarone, dronederone or propafenone may be used to treat or prevent antiarrythmia. In another embodiment, pharmaceutical compositions containing ziprasidone or risperidone may be used to treat or prevent psychotic conditions. In still another embodiment, pharmaceutical compositions containing dopamine agonists (e.g., carbidopa, levidopa, etc.) may be used too treat or prevent Parkinson's disease, etc. In still another embodiment, pharmaceutical compositions containing antihypertensive agents (e.g., acebutolol, atenolol, betaxolol, bisoprolol, bucindolol, carvedilol, dilevalol, labetalol, esmolol, etoprolol, nadalol, nevibulol, oxprenolol, propanolol, sotalol) may be used to treat or prevent cardiovascular disease. In still another embodiment, pharmaceutical compositions containing cilostazol may be used to treat or prevent various cardiovascular conditions, including cerebral ischemia, restenosis, bradychardia, peripheral arterial disease, intermittent claudication, critical limb ischemia and dyslipidemia. In still another embodiment, pharmaceutical compositions containing cilostazol may be used to treat or prevent cardiovascular conditions, including cerebral ischemia, restenosis, bradychardia, peripheral arterial disease, intermittent claudication, critical limb ischemia and dyslipidemia without the headaches and palpitation associated with immediate release cilostazol compositions.

### 5.4 Methods of Administration and Doses

The pharmaceutical compositions described herein may be advantageously used in human medicine. As previously described in Section 5.3 above, the pharmaceutical compositions described are useful for the treatment or prevention of various diseases.

When used to treat or prevent the above diseases or disorders, pharmaceutical compositions may be administered or applied singly, or in combination with other agents. Pharmaceutical compositions may also be administered or applied singly, in combination with other pharmaceutically active agents.

The current invention provides methods of treatment and prophylaxis by administration to a patient in need of such treatment of a therapeutically effective amount of a pharmaceutical composition of the invention. The patient may be an animal, more preferably, is a mammal and most preferably, is a human.

The pharmaceutical compositions of the invention, which comprise one or more drugs, are preferably administered orally. The pharmaceutical compositions of the invention may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc) that can be used to administer pharmaceutical composition of the invention. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner and will depend in-part upon the site of the medical condition. In most instances, administration will result in the release of the pharmaceutical compositions of the invention into the bloodstream.

In specific embodiments, it may be desirable to administer one or more pharmaceutical composition of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of the disease.

In certain embodiments, it may be desirable to introduce one or more pharmaceutical compositions of the invention into the central nervous system by any suitable route, including intraventricular, intrathecal and epidural injection. Intraventricular injection may be facilitated by an intraventricular catheter, for example, attached to a reservoir, such as an Ommaya reservoir.

In another embodiment, the pharmaceutical compositions of the invention can be delivered in a vesicle, in particular a liposome (See, Langer, 1990, Science, 249:1527-1533; Treat et al., in “Liposomes in the Therapy of Infectious Disease and Cancer,” Lopez-Berestein and Fidler (eds.), Liss, New York, pp.353-365 (1989); see generally “Liposomes in the Therapy of Infectious Disease and Cancer,” LopezBerestein and Fidler (eds.), Liss, N.Y., pp.353-365 (1989)).

The amount of drug that will be effective in the treatment or prevention of a disease in a patient will depend on the specific nature of the condition, and can be determined by standard clinical techniques known in the art. In addition, in vitro or in vivo assays may optionally be employed to help identify optimal dosage ranges. The amount of a drug administered will, of course, be dependent on, among other factors, the subject being treated, the weight of the subject, the severity of the affliction, the manner of administration and the judgment of the prescribing physician.

The amount and type of a drug, solubilizer and release modulator included in a specific pharmaceutical composition may vary according to the knowledge of one of ordinary skill in the art in view of the particular other components of the pharmaceutical composition and the specific therapeutic effects desired.

However, in one embodiment, the amount of a drug may be from about 0.25 w/w to about 80% w/w of the pharmaceutical composition. In another embodiment, the amount of a drug may be from about 0.5% w/w to about 50% w/w of the pharmaceutical composition. In yet another embodiment, the amount of a drug may be may be from about 0.75% w/w to about 24% w/w of the pharmaceutical composition.

In one embodiment, the amount of solubilizer used may be from about 5% w/w to about 99% w/w of the pharmaceutical composition. In another embodiment, the amount may be from about 15% w/w to about 95% w/w of the pharmaceutical composition. In yet another embodiment, the amount may be from about 30% w/w to about 95% w/w of the pharmaceutical composition. In yet another embodiment the relative amounts of the solubilizer to drug in the composition may be from about 1:1 to about 1:10.

In one embodiment, the amount of release modulator used may be from about I % w/w to about 50% w/w of the pharmaceutical composition. In another embodiment, the amount may be from about 5% w/w to about 30% w/w of the pharmaceutical composition. In yet another embodiment, the amount may be from about 10% w/w to about 20% w/w of the pharmaceutical composition Preferably, the dosage forms are adapted to be administered to a patient no more than twice per day, more preferably, only once per day. Dosing may be provided alone or in combination with other drugs and may continue as long as required for effective treatment or prevention of the disease.

### 5.5 Combination Therapy

In certain embodiments, the pharmaceutical compositions of the invention can be used in combination therapy with at least one other therapeutic agent. The pharmaceutical composition of the invention and the therapeutic agent can act additively or, more preferably, synergistically. In one embodiment, pharmaceutical composition of the invention is administered concurrently with the administration of another therapeutic agent. In another embodiment, a pharmaceutical composition of the invention is administered prior or subsequent to administration of another therapeutic agent.

### 6. EXAMPLES

The invention is further defined by reference to the following examples, which describe in detail, various pharmaceutical compositions of the invention. It will be apparent to those skilled in the art that many modifications, both to materials and methods, may be practiced without departing from the scope of the invention.

### 6.1 Example 1

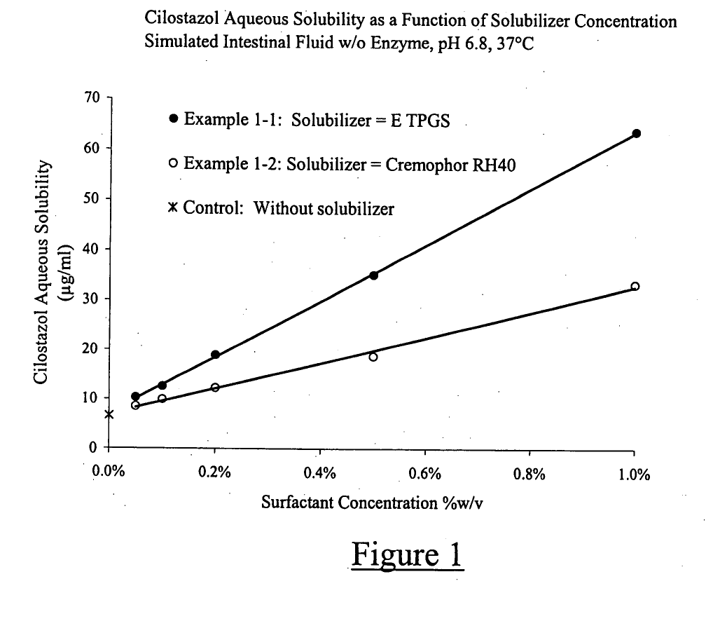

Example 1 illustrates enhancement of the aqueous solubility of cilostazol with two representative solubilizers: a tocol derivative (Vitamin E Polyethylene Glycol Succinate, NF, or d-a-tocopherol polyethylene glycol 1000 succinate; Vitamin E TPGS, Eastman Chemical Co.) [Example 1-1] and a polyethoxylated fatty acid derivative, (Polyoxyl 40 Hydrogenated Castor Oil, NF, Cremophor RH40; BASF) [Example 1-2]. Solutions of simulated intestinal fluid without enzyme (USP 26, pH 6.8) were prepared over a range of solubilizer concentrations. Excess cilostazol was added and equilibrated. with gentle mixing at controlled temperature (37+0.5° C.). The aqueous solutions with excess drug were then filtered (0.2 g nominal pore size) and the clear filtrate was diluted and assayed by HPLC for cilostazol concentrations. Results are shown in FIG. 1.

The intrinsic solubility of cilostazol under these conditions was 6.5 fig/ml, and solubility increased linearly with solubilizer concentration over the range tested. When d-a-tocopherol polyethylene glycol 1000 succinate was the solubilizer, the increase in solubility of cilostazol over its intrinsic aqueous solubility ranged from about a 60% increase at 0.05% w/v aqueous solubilizer concentration to about a 10-fold increase at I % w/v aqueous solubilizer concentration. When Cremphor RH40, was the solubilizer, the solubility enhancement of cilostazol ranges from about a 30% increase at 0.05% w/v solubilizer concentration to about a 5-fold increase at 1% w/v aqueous solubilizer concentration.

Solubility enhancement for several additional solubilizers and mixtures of solubilizers are shown in the table below.

| | | Solubilizer | Cilostazol |
|---|---|---|---|
| | | Aqueous | Aqueous |
| | | Concentration | Concentration |
| Example | Solubilizer | (% w/v) | (μg/ml) |
| Control | No solubilizer | 0% | 6.5 |
| 1-3 | Polysorbate 80 | 0.1% | 9.6 |
| 1-4 | d-alpha-tocopherol polyethylene | 0.1% | 15.8 |
| | glycol 1000 succinate/dl-alpha | | |
| | tocopherol/medium chain | | |
| | monoglycerides (Capmul | | |
| | MCM)/ethanol [2:1:2:1 ratio] | | |
| 1-5 | Polyoxyl 35 Castor Oil/Polyoxyl 40 | 0.2% | 20.3 |
| | Hydrogenated Castor Oil/Polysorbate | | |
| | 80lLabrasol/medium chain | | |
| | monoglycerides (3:3:3:9:2 ratio) | | |
| 1-6 | d-alpha-tocopherol polyethylene | 0.3% | 32.7 |
| | glycol 1000 succinate/dl-alpha | | |
| | tocopherol (4:1 ratio) | | |
| 1-7 | d-alpha-tocopherol polyethylene | 0.3% | 31.2 |
| | glycol 1000 succinate/dl-alpha | | |
| | tocopherol succinate (4:1 ratio) | | |
| 1-8 | Cremophor RH40/d-alpha tocopherol1 | 1.2% | 57.3 |
| | succinate (3:2 ratio) | | |
| 1-9 | Cremophor EL/d-alpha tocopherol | 1.2% | 29.9 |
| | succinate (3:2 ratio) | | |
| 1-10 | Polyoxyl 35 Castor Oil/Acetylated | 4% | 79 |
| | Monoglycerides/Polyvinylpyrrolidone K30* | | |
| | (1:1:1 ratio) | | |
| 1-11 | Polysorbate 80/Sorbitan monoleate | 9% | 116 |
| | (2:1 ratio) | | |

*spray-dried solid dispersion from isopropanol solution with cilostazol at 8% w/w in dried powder.

### 6.2 Example 2

Example 2 illustrates synchronized solubilizer and cilostazol release from dosage forms prepared according to the current invention. Dosage forms were prepared with a solubilizer (i.e., d-a-tocopherol polyethylene glycol 1000 succinate (Vitamin E TPGS, Eastman Chemical Company)), a release modulator (Le., d-a-tocopherol succinate, (Spectrum Chemical Co.)) and an additive ((i.e., polyethylene glycol 8000 (Spectrum Chemical Co.)). The compositions of the prepared dosage form are summarized below.

| | Compositions |
|---|---|
| | mg/dosage form |
| Component | 2-1 |
| Cilostazol | 125 |
| d-alpha tocopherol | 572 |
| polyethylene glycol | |
| 1000 succinate | |
| d-alpha tocopherol | 64 |
| succinate | |
| Polyethylene glycol | 52 |

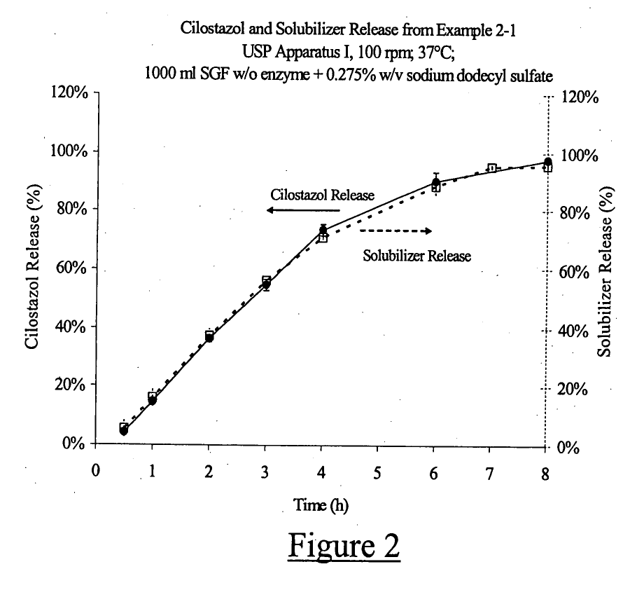

All components except the drug were melted, then the drug was added and the mixture homogenized briefly with a high-shear rotor-stator homogenizer. The molten mixture was filled into hard-gelatin capsules and allowed to congeal at uncontrolled room temperature (−25° C.). The resulting capsules were tested in a USP apparatus 1 at 100 rpm with a dissolution medium consisting of 1,000 ml of simulated gastric fluid without enzyme (USP 26) containing 0.275% w/w sodium dodecyl sulfate. The dissolution of the drug, d-alpha-tocopherol polyethylene glycol 1000 succinate, and d-alpha-tocopherol succinate were monitored by HPLC. The dissolution profile as a function of time for both the drug and the solubilizers are shown in FIG. 2. Release of both the drug and the solubilizer are synchronized, with a correlation coefficient greater than 0.99 over the 8 hour release period.

### 6.3 Example 3

Example 3 illustrates synchronized release of cilostazol and solubilizer from two additional dosage forms prepared according to the current invention. Dosage forms were prepared using a solubilizer (i.e., d-alpha-tocopherol polyethylene glycol 1000 succinate), a release modulator, (i.e., dl-a-tocopherol (Spectrum Chemical Co.)), and a solvent (i.e., acetic acid (Spectrum Chemical Co.)). The compositions of the prepared dosage forms are summarized below.

| | Compositions |
|---|---|
| | mg/dosage form |
| Component | 2-1 |
| Cilostazol | 125 |
| d-alpha tocopherol | 338 |
| polyethylene glycol | |
| 1000 succinate | |
| dl-alpha tocopherol | 84 |
| Acetic Acid | 219 |

All components except the drug and acetic acid were melted and blended. The drug was dissolved in the acetic acid, then added to the other molten components. After vortex mixing, the molten solution was filled into hard-gelatin capsules and 5 allowed to congeal at room temperature (−25° C.).

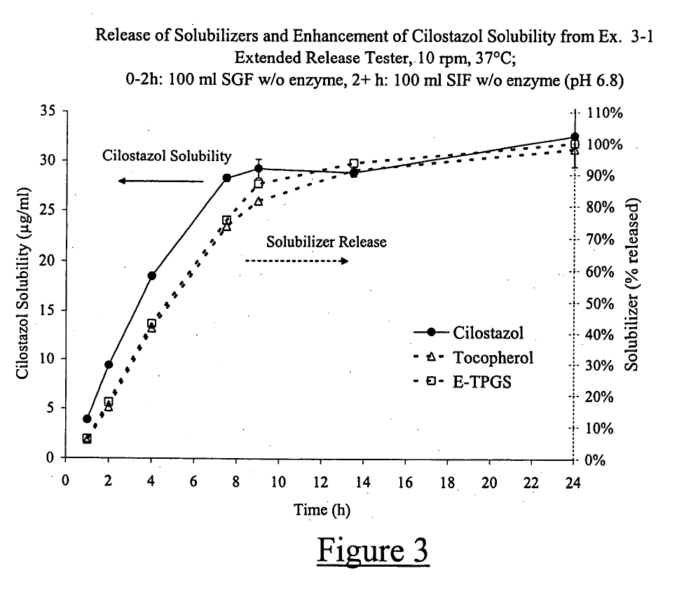

The dosage forms were tested in a dissolution experiment in which the dosage form was repeatedly exposed to a non-solubilizing dissolution media after selected time intervals. The dissolution experiment utilized a rotating bottle apparatus (Extended Release Tester; VanKel) at 10 rpm, 3710.1 ° C. with 100 ml simulated gastric fluid without enzyme (USP 26) for the first 2 hours, replaced with 100 ml simulated intestinal fluid without enzyme (USP 26, pH 6.8) thereafter. Dissolution of drug, d-alpha-tocopherol polyethylene glycol 1000 succinate, and dl-alpha tocopherol were monitored by HPLC. FIG. 3 shows the release of d-alpha-tocopherol polyethylene glycol 1000 succinate and di-alpha tocopherol and the increase in cilostazol solubility. The release of the solubilizer, d-alpha-tocopherol polyethylene glycol 1000 succinate, and the release modulator, di-alpha tocopherol, exhibited were synchronized with the drug release (correlation coefficient >0.98 over the −13 hour release period between drug and both the solubilizer and the release modulator). Cilostazol solubility was increased throughout the release period, resulting in an overall increase of about 5-fold relative to the intrinsic solubility.

### 6.4 Example 4

Example 4 illustrates the effect of varying the concentration of a release modulator, (i.e., dl-alpha tocopherol succinate,) in compositions prepared according to the current invention using d-alpha tocopherol polyethylene glycol 1000 succinate as a solubilizer. The compositions of the prepared dosage forms are summarized below.

Compositions
(mg/dosage form)

| Component | 4-1 | 4-2 | 4-3 | 4-4 |
|---|---|---|---|---|
| Cilostazol | 50 | 50 | 50 | 50 |
| d-alpha tocopherol | 430 | 387 | 344 | 301 |
| polyethylene glycol | | | | |
| 1000 succinate | | | | |
| d-alpha tocopherol | 0 | 43 | 86 | 129 |
| succinate | | | | |
| Polyethylene glycol | 20 | 20 | 20 | 20 |

All components except the drug were melted, then the drug and HPMC were added and the mixture homogenized briefly with a high-shear rotor-stator homogenizer. The molten mixture was filled into hard-gelatin capsules and allowed 5 to congeal at uncontrolled room temperature (−25° C.).

The dosage forms were tested in a dissolution experiment in which the dosage form was repeatedly exposed to a non-solubilizing dissolution media after selected time intervals. This experiment utilized a rotating bottle apparatus (Extended Release Tester, VanKel) at 10 rpm, 370.1° C. with 100 ml simulated gastric fluid without enzyme (USP 26) for the first 2 hours, replaced with 100 ml simulated intestinal fluid without enzyme (USP 26, pH 6.8) thereafter. Drug and d-alpha tocopherol polyethylene glycol 1000 succinate dissolution were monitored by HPLC. The time to 70% dissolution is summarized in the table below.

| | d-alpha tocopherol | |
|---|---|---|
| | succinate concentration in | |
| Composition | dosage form (% w/w) | Time to 70% release |
| 4-1 | 0 | <1 h |
| 4-2 | 8.6% | 5 h |
| 4-3 | 17.2% | 8 h |
| 4-4 | 25.8% | 15 h |

The time to 70% release for Compositions 4-2 through 4-4 increased exponentially with release modulator concentration.

### 6.5 Example 5

Example 5 illustrates synchronized solubilizer and cilostazol release from dosage forms prepared according to the current invention, using the solubilizers, d alpha-tocopherol polyethylene glycol 1000 succinate and Linoleoyl Macrogolglycerides (Labrafil 2125CS). The release modulators were Glycerol Dibehenate (Compritol 888 Ato, Gattefosse) and/or hydroxypropylmethylcellulose (Methocel KIOOM, Dow Chemical Company). The compositions of the prepared dosage forms are summarized below.

Compositions
(mg/dosage form)

| Component | 5-1 | 5-2 | 5-3 | 5-4 |
|---|---|---|---|---|
| Cilostazol | 50 | 50 | 50 | 50 |
| Linoleoyl | 377 | 296 | 316 | 307 |
| Macrogolgycerides | | | | |
| (Labrafil 2125CS) | | | | |
| Polyethylene Glycol 8000 | 20 | 16 | 0 | 0 |
| Glyceryl Dibehenate | 0 | 0 | 90 | 135 |
| (Compritol 888 Ato) | | | | |
| HPMC K100M | 43 | 130 | 36 | 0 |

All components except the drug and HPMC were melted, then the drug and HPMC were added and the mixture homogenized briefly with a high-shear rotorstator homogenizes. The molten mixture was filled into hard-gelatin capsules and allowed to congeal at uncontrolled room temperature (−25° C.). 10 The dosage forms were tested in a dissolution experiment in which the dosage form was repeatedly exposed to a non-solubilizing dissolution media after selected time intervals. This experiment utilized a rotating bottle apparatus (VanKel Extended Release Tester) at 10 rpm, 370.1° C. with 100 ml simulated gastric fluid without enzyme (USP 26) for the first 2 hours, replaced with 100 ml simulated intestinal fluid without enzyme (USP 26, pH 6.8) thereafter. Drug and d-alpha-tocopherol polyethylene glycol 1000 succinate dissolution were monitored by HPLC. The cilostazol aqueous solubility was enhanced throughout the extended release period indicating synchronized release of the drug and solubilizer. The table summarizes solubilizer release time as well as the increase in cilostazol aqueous solubility relative to the intrinsic solubility.

| | | | Increase in |
|---|---|---|---|
| | Time to 50% | Time to 100% | Cilostazol |
| Composition | Solubilizer Release | Solubilizer Release | Solubility |
| 5-1 | 1.2 h | 4.4 h | 2.3 X |
| 5-2 | 10.8 h | 23 h | 2.3 X |
| 5-3 | 2.0 h | 4.2 h | 2.2 X |
| 5-4 | 1.2 h | 3.8 h | 2.2 X |

### 6.6 Example 6

Example 6 shows the performance of dosage forms prepared according to the current invention using Polyoxyl 40 Hydrogenated Castor Oil NF (Cremophor RH40, BASF) as the solubilizer and hydroxypropyl methylcellulose (HPMC K4M, ) as the release modulator. The compositions of the prepared dosage forms are summarized below.

Compositions
(mg/dosage Form)

| Component | 6-1 | 6-2 |
|---|---|---|
| Cilostazol | 25 | 25 |
| Cremo hor RH40 | 125 | 125 |
| HPMC K4M | 85 | 85 |
| Talc | 9 | 9 |
| Colloidal Si02 | 1 | 1 |
| Polyvinylpyrrolidone | 45 | 45 |
| K90 | | |
| Sodium dodecyl sulfate | — | 2.5 |

A binding solution of polyvinylpyrrolidone K90, Cremophor RH40, dehydrated alcohol USP, and deionized water was prepared and allowed to shake until all of the polyvinylpyrrolidone dissolved. Cilostazol was blended with talc, colloidal SiO2 and the wetting agent, sodium dodecyl sulfate (Composition 3-2) and then passed through a 60 MESH screen. The microcrystalline cellulose and HPMC K4M were then added and blended in a polybag for −20 minutes. The resulting powder was needed with the binder solution and the dough was extruded through the barrel of a 10 ml syringe. The extruded material was dried at 25° C./26-30% RH for about 20 hours. The dried extrusion was cut into pellets about 3-5 mm in length and filled into hard-gelatin capsules.

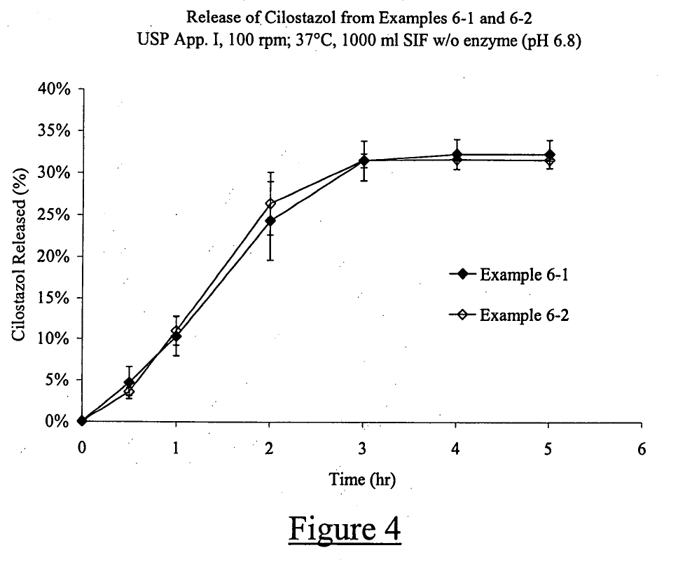

The capsules were tested in a USP apparatus I at 100 rpm, 37.Ot0.5° C., with a dissolution medium consisting of 1,000 ml of simulated gastric fluid without enzyme (USP 26). The dissolution of cilostazol as a function of time is shown in FIG. 4. The compositions reached a plateau at about 3 hours, with an increase in the cilostazol solubility of about 30%.

### 6.7 Example 7

A tablet dosage form according to the present invention was prepared with d alpha-tocopherol polyethylene glycol 1000 succinate as a solubilizer and HPMC as a release modulator. The composition of the tablets is shown below.

| | Compositions |
|---|---|
| | (mg/dosage Form) |
| Component | 7-1 |
| Cilostazol | 50 |
| d-alpha tocopherol | 200 |
| polyethylene glycol | |
| 1000 succinate | |
| HPMC K4M | 60 |
| Microcrystalline Cellulose | 80 |
| (Avicel pH 113) | |
| Starch 1500 | 100 |
| Talc | 12 |
| Polyvinylpyrrolidone | 40 |
| K90 | |
| Magnesium Stearate | 10 |

Cilostazol was blended with ½ the talc and Starch 1500, then passed through a #100 MESH screen. Additionally 1/2 the HPMC and microcrystalline cellulose and ¼ the polyvinylpyrrolidone were mixed and passed through the same 100 MESH screen. The two mixtures were then combined and mixed well.

Separately, d-alpha-tocopherol polyethylene glycol 1000 succinate and magnesium stearate were mixed for 15-20 minutes. Then ½ the talc was added and the mixing continued for 5 minutes. Finally, 1/2 the MCC, HPMC, Starch 1500 and ¾ the PVP were added and mixed for 10-15 minutes. The drug-containing blend and the d-alpha-tocopherol polyethylene glycol 1000 succinate-containing blend were mixed in a polybag for about 20 minutes.

The final blend was compressed into tablets using a Carver press using IR pellet disks (12.5 mm diameter) at a force of 2,500 lb for 1-2 sec.

### 6.8 Example 8

Example 8 shows the enhancement of the solubility of the weakly basic antihypertensive, carvedilol, using various solubilizers in accordance with the present invention. The solubilizers were a polyethoxylated castor oil derivative (polyoxyl 35castor oil, NF; Cremophor® EL, BASF), a tocol derivative (d-alpha tocopherol polyethylene glycol 20 1000 succinate, Vitamin E TPGS®, Eastman Chemical Co.), a 5 polyethoxylated fatty acid derivative (linoleyl macrogolglycerides, EP, Labrafil 2125CS, Gattefosse). Composition 8-4 also includes a fatty acid derivative (Glycerol Dibehenate; Compritol 888 Ato, Gattefosse). A control of carvedilol with no solubilizer was also prepared.

| | | Composition |
|---|---|---|
| Example | Components | (% w/w) |
| 8-1 | Cremophor EL | 94.0% w/w |
| | Carvedilol | 6.0% |
| 8-2 | E-TPGS | 94.0% w/w |
| | Carvedilol | 6.0% |
| 8-3 | Cremophor EL | 75.2% w/w |
| | Labrafil 2125CS | 18.8% |
| | Carvedilol | 6.0% |
| 8-4 | E-TPGS | 75.2% w/w |
| | Compritol 888 Ato | 18.8% |
| | Carvedilol | 6.0% |
| Comparative | | |
| Example | Components | Composition |
| Control | Carvedilol | 100% w/w |

Formulations 8-1 and 8-2 were prepared by dissolving carvedilol base at 60 mg/g in the liquid excipients at room temperature. Formulations 8-3 and 8-4 were prepared by dissolving carvedilol base at 60 mg/g in the molten excipient mixture at about 80° C. and cooling the resulting clear liquid at ambient temperature to obtain a 15 solid.

In order to determine solubility and release properties, all compositions were dispersed in simulated gastric fluid without enzyme (pH 1.210.1, USP 26); in simulated intestinal fluid without enzyme at pH 6.8 (USP 26); or in simulated intestinal fluid without enzyme at pH 8. Formulations 8-1 through 8-4 were dispersed at 5X dilution (final carvedilol concentration 12 mg/ml) and the control was dispersed at 12 mg/ml final carvedilol concentration. The resulting dispersions were mixed on a rotator for 4 hours at 37±1° C. Carvedilol concentration in the aqueous phase was determined by filtering the dispersion through an 0.2 p Nylon filter, diluting the filtrate 1 to 1 with acetonitrile and assaying the diluted filtrate by reversed-phase HPLC using a 4.6×150 mm column with a 5p C8 stationary phase. The mobile phase was a gradient with acetonitrile/20 mM phosphate (pH 2.3) at 1.2 ml/min. The measured carvedilol concentrations are shown in the table below

Concentration of carvedilol in aqueous phase after 4 hours at 37° C.

| | Example | | | | |
|---|---|---|---|---|---|
| | 8-1 | Example 8-2 | Example 8-3 | Example 8-4 | Control |
| | (mg/ml) | (mg/ml) | (mg/ml) | (mg/ml) | (mg/ml) |
| SGF, | 10.7 | 9.1 | 11.3 | 7.9 | 0.36 |
| pH 1.2 | | | | | |
| SIF, | 9.1 | 8.9 | 10.8 | 7 | 0.044 |
| pH 6.8 | | | | | |
| SIF, | 8.9 | 8.8 | 11.0 | 7.8 | 0.008 |
| pH 8 | | | | | |

As can be seen, the carvedilol dissolution/solubility at 4 hours increases with decreasing pH, consistent with formation of more of the water-soluble protonated carvedilol species. In pH 1.2 SGF, where the drug would be expected to be essentially completely ionized, the dissolved drug concentration is nevertheless fairly low due to formation of the acid addition HCI salt which has an equilibrium solubility of only about 1 mg/ml.

For Examples 8-1 through 8-4 prepared according to the present invention, the carvedilol solubility is dramatically increased and there is little difference between the dissolved drug concentrations in the various media at different pH values. For Example 8-2 there is less than 4% difference between the solubility obtained in pH 8 SIF (8.8 mg/ml) and pH 1.2 SGF (9.1 mg/ml), while for Example 8-1, there is less than 20% difference (10.7 mg/ml in SGF vs. 8.9 mg/ml in pH 8 SIF). These results show that with solubility enhancement using the current invention, cilostazol solubility becomes substantially independent of the pH of the media and is also not affected by the presence of chloride ions.

### 6.9 Example 9

A tablet dosage form according to the present invention was prepared containing carvedilol with d-alpha-tocopherol polyethylene glycol 1000 succinate as the solubilizer. Release modulators were a fatty acid derivative (Glycerol Dibehenate, S Compritol 888 Ato, Gattefosse), a cellulose derivative (HPMC KIOOLV and HPMC K4MP, Dow Chemical Co.) and a polyacrylic (Carbopol 940, BF Goodrich) were used as the release modulators. The composition of the tablets is shown below.

Compositions (mg/dosage form)

| Component | 9-1 | 9-2 |
|---|---|---|
| Carvedilol | 25 | 25 |
| d-alpha tocopherol | 221 | 210 |
| polyethylene glycol | | |
| 1000 succinate | | |
| (Vitamin E TPGS) | | |
| Glycerol Dibehenate | 55 | 53 |
| (Compritol 888 Ato) | | |
| HPMC K100LV | 59 | — |
| HPMC K4MP | 59 | 56 |
| Carbopol 940 | — | 56 |
| Amorphous Silica | 1 | 1 |
| (Cab-O-SiI M5) | | |

Compritol and Vitamin E TPGS were dry blended in an Osterizer blender, then the polymers and silica were added and blended in 4 stages. The resulting mixture was sieved and the <60 MESH fraction collected. Carvedilol was added and the powder mixed for 8 hours on a wrist-action shaker with periodic mixing with a spatula (−1/hour).

The final blend was compressed into tablets using a Carver press using IR pellet disks (12.5 mm diameter) at a force of 2,500 lb for 1-2 sec. The tablets were tested in a USP apparatus I at 100 rpm, 37.OfO.5° C. The dissolution medium was 1,000 ml simulated gastric fluid without enzyme (USP 26) for the first 2 hours, which was then replaced with 1,000 ml simulated intestinal fluid without enzyme for the remainder of the 24 hour experiment. Dissolution of carvedilol and the solubilizer Vitamin E TPGS were analyzed using an Agilent UV/Vis spectrophotometer with an on-line sample collection valve. Assay of carvedilol was based on absorbance at 360 nm and assay of Vitamin E TPGS was based on absorbance at 285 nm after subtraction of the carvedilol absorbance at this wavelength. Quantification was by linear regression of external standards of known carvedilol and Vitamin E TPGS concentration.

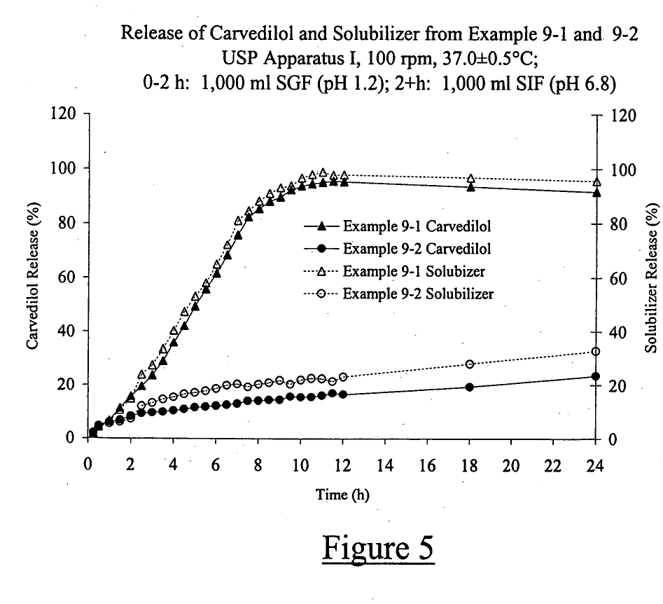

The dissolution profile as a function of time for both the drug and the solubilizer are shown in FIG. 5. Example 9-1 showed an extended release profile with time to complete release -1 I h, and the release of drug and the solubilizer were well synchronized throughout the 0-11 hour period (r>0.99). Example 9-2 had an extended release profile with time to complete release >24 h. The drug and solubilizer release were synchronized throughout the 0-24 hour experimental period (r>0.97).

### 6.10 Example 10

A synchronized solubilizer release composition in accordance with the present invention was prepared using a tocol derivative as a solubilizer (Vitamin E-TPGS, Eastman Chemical Company), a fatty acid derivative as a release modulator (Compritol 888 Ato, Gattefosse), and carvedilol in the proportions 75.2/18.8/6.0% w/w. Vitamin E-TPGS and Compritol 888 were melted and blended together at 80° C., then carvedilol free base was dissolved in the mixture. The molten solution was filled into Size 3 hard-gelatin capsules at a fill weight of 0.21 mg/capsule (12.5 mg carvedilol/capsule) and allowed to solidify at ambient temperature (Example 10-1). Dissolution of carvedilol from these capsules was tested using 2 capsules each (25 mg carvedilol total) in a rotating bottle apparatus (Extended Release Tester; VanKel) at 10 rpm and 37±0.1° C. Dissolution media were 100 ml SGF without enzyme (pH 1.2, USP 26) or in 100 ml SIF without enzyme (pH 6.8, USP 26). A comparator formulation without synchronized solubilizer release was also tested under the same conditions (Comparator 10-1; Coreg® 25 mg carvedilol tablet; GlaxoSmithkline). Carvedilol release as a function of time was monitored as described in Example 8.

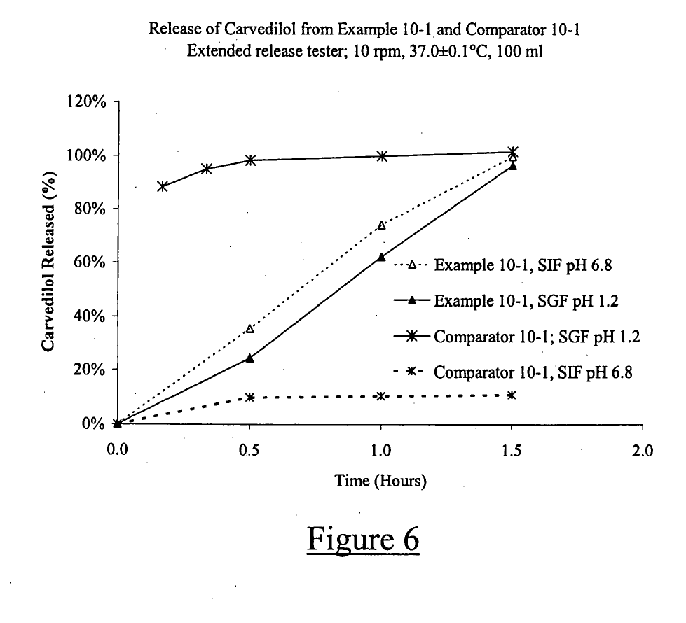

The resulting dissolution profiles are shown in FIG. 6. As can be seen, Example 9-1 exhibits both enhanced solubility and extended release with less than <40% of drug dissolved 0.5 hours and >80% dissolved by 0.5 hours in both pH 1.2 SGF and in pH 6.8 SIF. The comparator 9-1 releases 100% in pH 1.2 SGF by 0.5 h and releases only −20% by 1.5 hours in SIF due to the limited solubility of the drug at this pH.

### 6.11 Example 11

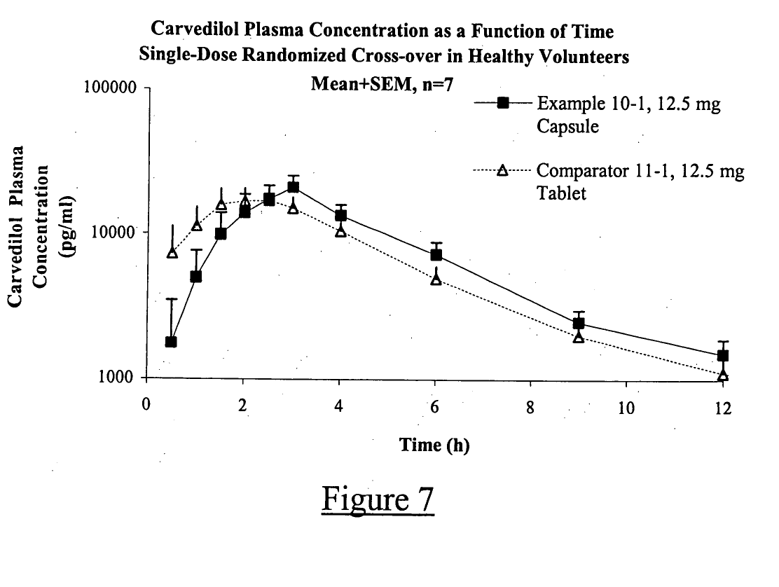

The synchronized solubilizer release dosage form in Example 10 (Example 10-1) was dosed in a randomized, single-dose cross-over study in 7 healthy volunteers with a commercial immediate release tablet as a comparator (Comparator I 1-1; Coreg® 12.5 mg carvedilol tablet; GlaxoSmithkline). Both treatments were administered immediately after breakfast. Blood samples of about 7 ml were collected in EDTA tubes, centrifuged, and the plasma assayed for carvedilol using a validated LC/MS/MS method. FIG. 7 shows the resulting plasma profiles and the table below shows the summary pharmacokinetic parameters calculated using standard non-compartmental techniques. Maximum plasma concentration and time to maximum plasma concentration were taken directly from the data. Tag was calculated by extrapolation of the straight line from the initial absorption curve. The area under the curve (AUC) value from 0-0o was calculated by trapezoidal integration. The capsule of the current example showed a consistent delayed release profile with a mean lag-time of 1.2 hours and a TmaX range of 1.5-3 hours. The comparator immediate release tablet had a highly variable initial absorption with a mean lag time of 0.5 hours and a TmaX range of 0.5-3 hours. As shown in the table below, the AUCo_ ratios show that bioavailability was significantly increased due to the synchronized and enhanced solubilization of the drug.

| | | | Within-Subject |
|---|---|---|---|
| | Formulation 10-1 | Comparator 11-1 | Ratios |
| Parameter | Mean (SEM | Mean (SEM) | Mean [90% CI] |
| Cmax | 31,137 (5,565) | 23,274 (4,055) | 138% [99-191% j |
| (pg/tn l) | | | |
| AUCo | 125.1 (23.7) | 108.9 (23.5) | 118% [102-137%] |
| (n * h/ml) | | | |
| key (h\hat{ }-1) | 0.201 (0.031) | 0.224 (0.042) | 93% [71-121%] |
| Tea (h) | 1.2 (0.3 | 0.5 (0.2) | Mean |
| | | | Difference = +0.7 h |
| Tmax (h) | 2.2 (0.3) | 1.9 (0.3) | Mean |
| | | | Difference = +0.4 h |

### 6.12 Example 12

Additional compositions according to the present invention comprising zafirlukast are described below. These were prepared by dissolving zafirlukast in the molten excipient or excipient mixture at elevated temperature, then allowed to cool down and to form a solid plug. To prepare a dosage form for testing, 200 mg of the molten composition was filled in size 3 two-piece hard gelatin capsules for unit strength of 10 mg zafirlukast.

### Example 12-1

| Compositions | (w/w) |
|---|---|
| Zafirlukast | 5 |
| TPGS | 76 |
| Glycerol Dibehenate (Compritol 888) | 19 |

### Example 12-2

| Compositions | (w/w) |
|---|---|
| Zafirlukast | 5 |
| TPGS | 57 |
| Glycerol Dibehenate (Compritol 888) | 38 |

### Example 12-3

| Compositions | (w/w) |
|---|---|
| Zafirlukast | 5 |
| TPGS | 57 |
| Glycerol Dibehenate (Compritol 888) | 19 |
| Glycerol Distearate (Precirol ATO) | 19 |

### Example 12-4

| Compositions | (w/w) |
|---|---|
| Zafirlukast | 5 |
| TPGS | 76 |
| Vitamin E succinate | 19 |

### Example 12-5

| Compositions | (w/w) |
|---|---|
| Zafirlukast | 5 |
| Gelucire 44/14 | 76 |
| Glycerol Dibehenate (Compritol 888) | 19 |

### Example 12-6

| Compositions | (w/w) |
|---|---|
| Zafirlukast | 5 |
| Gelucire 44/14 | 76 |
| Glycerol Distearate (Precirol ATO) | 19 |

### Example 12-7

| Compositions | (w/w) |
|---|---|
| Zafirlukast | 5 |
| Cremophor RH40 | 76 |
| Glycerol Dibehenate (Compritol 888) | 19 |

### Example 12-8

| Compositions | (w/w) |
|---|---|
| Zafirlukast | 5 |
| Cremophor RH40 | 76 |
| Glycerol Distearate (Precirol ATO) | 19 |

### 6.13 Example 13

Compositions described below were prepared by dissolving zafirlukast in the molten lipid excipient or lipid excipient mixture at elevated temperature. The HPMC polymer was then suspended in the molten composition to form a homogenous dispersion by homogenization or stirring, for example, at elevated temperature. The dispersion was filled in gelatin capsules to form a solid plug. The dispersion can also be extruded into desirable size and shape (granules by spheronization) and then filled in capsules.

Granules of zafirlukast, lipid excipient and HPMC can also be prepared separately or in any combination of the individual component, e.g., zafirlukast and TPGS without or without glycerol dibehenate, glycerol distearate or vitamin E succinate as solid solution or solid dispersion. The granules can be prepared with appropriate additives or blended with appropriate additives to be filled in capsules or compressed into pellets or tablets.

### Example 13-1

| Compositions | (w/w) |
|---|---|
| Zafirlukast | 5 |
| TPGS | 57 |
| Methocel K4M (HPMC) | 38 |

### Example 13-2

| Compositions | (w/w) |
|---|---|
| Zafirlukast | 5 |
| TPGS | 60 |
| Glycerol Dibehenate (Compritol 888) | 16 |
| Methocel K4M (HPMC) | 19 |

### Example 13-3

| Compositions | (w/w) |
|---|---|
| Zafirlukast | 5 |
| TPGS | 68 |
| Glycerol Distearate (Precirol ATO) | 8 |
| Methocel K4M (HPMC) | 19 |

### Example 13-4

| Compositions | (w/w) |
|---|---|
| Zafirlukast | 5 |
| TPGS | 68 |
| Glycerol Dibehenate (Compritol 888) | 8 |
| Glycerol Distearate (Precirol ATO) | 8 |
| Methocel K4M (HPMC) | 11 |

### Example 13-5

| Compositions | (w/w) |
|---|---|
| Zafirlukast | 5 |
| TPGS | 60 |
| Glycerol Dibehenate (Compritol 888) | 17 |
| Methocel K100LV (HPMC) | 19 |

### Example 13-6

| Compositions | (w/w) |
|---|---|
| Zafirlukast | 2 |
| TPGS | 55 |
| Vitamin E Succinate | 5 |
| Methocel K100LV (HPMC) | 38 |

### 6.14 Example 14

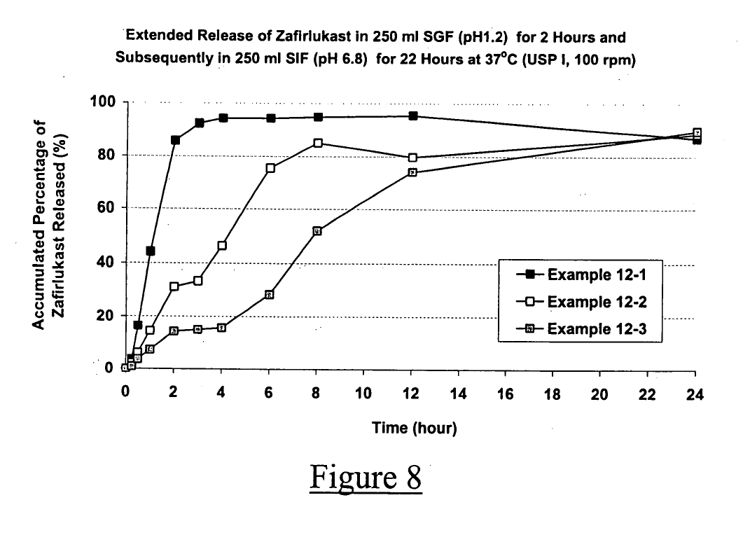

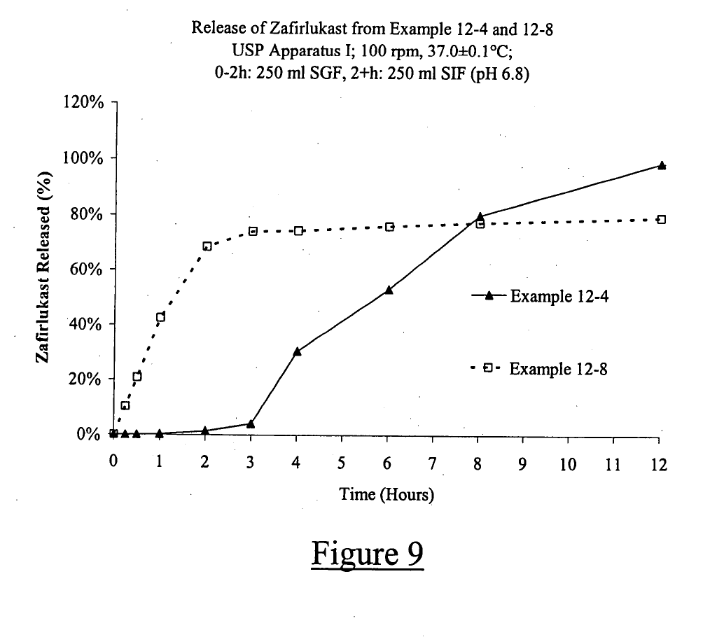

Dissolution of zafirlukast from capsules of Example 12 were performed to demonstrate the extended release and solubilization of zafirlukast over various period of times. Each capsule containing 10 mg zafirlukast in composition of examples 12 1, 12-2, 12-3, 12-4 and 12-8 was placed in a USP type I dissolution apparatus with 250 ml of pH 1.2 simulated gastric fluid without enzyme (100 rpm, 37° C.) for 2 hours. After 2 hours, the dissolution medium was replaced with 250 ml of pH 6.8 simulated intestinal fluid without enzyme and the dissolution study continued for another 22 hour. At given time points, an aliquot of the dissolution medium was sampled and assayed for the concentration of zafirlukast released (solubilized). The accumulated percentage of zafirlukast released from the capsules is summarized in FIG. 8 and 9 and represents more than 50-fold increase relative to the release of zafirlukast in the absence of solubilizers under these conditions.

### 6.15 Example 15

Compositions described below were prepared as follows. Granules of pioglitazone HCI, lipid excipient and HPMC were prepared separately with appropriate additives (Cab-O-Sil TS-530 amorphous fumed silica, 1% w/w), sieved to <60 MESH, and then blended together and compressed into tablets.

Example 15-1

| Compositions | (w/w) |
|---|---|
| Pioglitazone HCl | 5 |
| TPGS | 47.5 |
| Methocel K4M (HPMC) | 47.5 |

### Example 15-2

| Compositions | (w/w) |
|---|---|
| Pioglitazone HCl | 5 |
| TPGS | 47.5 |
| Methocel K100LV (HPMC) | 47.5 |

### Example 15-3

| Compositions | (w/w) |
|---|---|
| Pioglitazone HCl | 5 |
| TPGS | 47.5 |
| Methocel K100LV (HPMC) | 23.5 |
| Methocel E50 (HPMC) | 24 |

### 6.16 Example 16

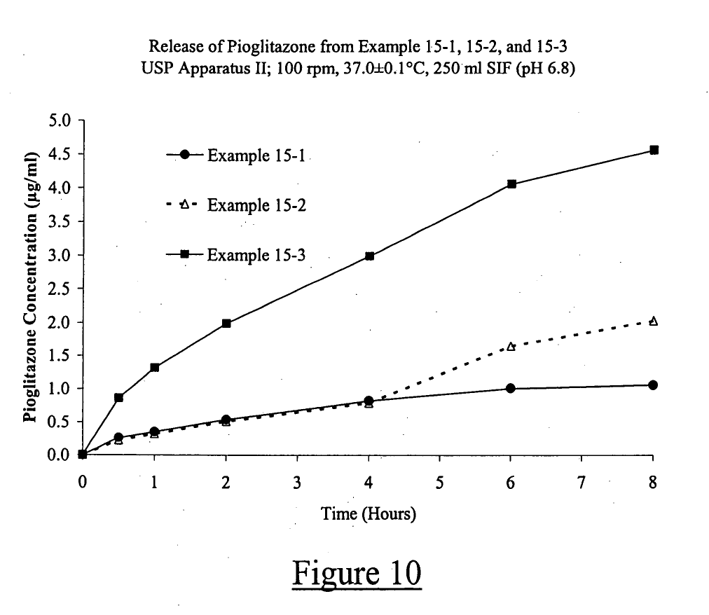

Dissolution of pioglitazone HCl tablets of Example 15 containing compositions from example 15-1 to 15-3 were performed to demonstrate the extended release and solubilization of pioglitazone over various period of times. Each tablet containing 50 mg pioglitazone HCI in composition of example 15-1 to 15-3 was placed in a USP type II dissolution apparatus, 100 rpm, with 250 ml of pH 6.8 simulated intestinal fluid without enzyme (100 rpm, 37° C.) for 8 hours. At given time points, an aliquot of the dissolution medium was sampled and assayed for the concentration of pioglitazone released (solubilized). The concentration of pioglitazone released as a function of time from the tablets is summarized in FIG. 10. Culmative increase in pioglitazone solubility over its intrinsic solubility at this pH ranges from about 36% increase for Example 15-1 to about 6-fold increase for Example 15-3. ranges.

### 6.17 Example 17

Compositions were prepared according to the present invention in which the poorly water-soluble basic drug carvedilol and solubilizers were separated in the dosage form.

Example 17-1

| | mg/capsule |
|---|---|
| Carvedilol + Release Modulator Granules | 194 |
| Carvedilol | 50 |
| Hydroxyproyl Methyl Cellulose K4M | 70 |
| Microcrystalline Cellulose | 25 |
| Starch | 20 |
| Polyvinyl Pyrrolidone K30 | 12 |
| Talc | 6 |
| Hydroxypropyl Methyl Cellulose E5 | 7 |
| Hydroxyropyl Methyl Cellulose E15 | 3 |
| Polyethylene Glycol 8000 | 1 |
| Solubilizer + Release Modulator Granules | 350 |
| a-d-tocopheryl Polyethylene glycol 1000 | 234 |
| Succinate (Vit E TPGS) | |
| Polyoxyl 40 Hydrogenated Castor Oil | 58 |
| (Cremophor RH40) | |
| Vitamin E Succinate | 58 |

Carvedilol pellets (−OS-1.0 mm diameter) containing components 1-6 were prepared in a manner similar to Example 7, then coated with components 7-9 in a fluid bed coater. The solubilizers and the release modulator (Vitamin E Succinate, alpha-tocopherol succinate) were melted and filled into hard-gelative capsules (Size 00). The drug+release modulator pellets were then added immediately while the fill was still molten. The capsules were then cooled at ambient temperature to produce a capsule exhibiting synchronized drug and solubilizer release containing a suspension of barrier-coated carvedilol pellets in the 10 solubilizer+release modulator matrix.

### Example 17-2

| | mg/dosage form |
|---|---|
| Carvedilol + Release Modulator Granules | .187 |
| Carvedilol | 50 |
| Hydroxyproyl Methyl Cellulose K4M | 65 |
| Microcrystalline Cellulose | 25 |
| Starch | 15 |
| Polyvinyl Pyrrolidone K30 | 12 |
| Talc | 4.4 |
| Magnesium Stearate | 1 |
| Colloidal Silicon Dioxide | 0.6 |
| Hydroxpropyl Methyl Cellulose E5 | 9 |
| Hydroxyropyl Methyl Cellulose E15 | 4 |
| Polyethylene Glycol 8000 | 1 |
| Solubilizer + Release Modulator Granules | 350 |
| a-d-tocopheryl Polyethylene glycol 1000 | 210 |
| Succinate (Vit E TPGS) | |
| Vitamin E Succinate | 35 |
| Microcrystalline cellulose | 70 |
| Colloidal silicon dioxide | 35 |

Carvedilol granules were prepared containing components 1-8, then coated in a fluid bed coater with components 9-11 to form barrier coated granules containing carvedilol and a release modulator. Solubilizer +release modulator granules were prepared separately. For example 17-2A, the carvedilol+release modulator granules were compressed first, followed by a second compression with the solubilizer granules to produce double-layered tablets with synchronized solubilizer and drug release. For example 1 7-2B, the drug +release modulator granules and the solubilizer+release modulator granules were blended and filled in Size 00 hard-gelatin capsules to produce a capsule with synchronized drug and solubilizer release.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the appended claims.

All publications and patents cited herein are incorporated by reference in their entirety.

What is claimed is:

1. A pharmaceutical composition comprising:
a therapeutically effective amount of a drug;
a solubilizer; and
a release modulator;
wherein the release of the drug and solubilizer are synchronized.

2. The pharmaceutical composition of claim 1, wherein the drug is pioglitazone, zafirlukast, simivastatin, atorvastin or fenofibrate.

3. The pharmaceutical composition of claim 1, wherein the drug is cilostazol.

4. The pharmaceutical composition of claim 1 or claim 3, wherein the solubilizer is a polyoxyethylene-polyoxypropylene block copolymer, a cyclodextrin or cyclodextrin derivative, a fatty acid derivative, a tocol derivative or mixtures thereof.

5. The pharmaceutical composition of claim 4, wherein the tocol derivative is a a-tocopherol ester, a polyethoxylated a-tocopherol ester or mixtures thereof.

6. The pharmaceutical composition of claim 4, wherein the tocol derivative is a-tocopherol, a-tocopherol acetate, a-tocopherol nicotinoate, a tocopherol succinate, a,-tocopherol polyethyleneglycol succinate, a-tocopherol polyethyleneglycol (200-8000 MW) succinate, a-tocopherol polyethylene glycol 400 succinate, a-tocopherol polyethyleneglycol 1000 succinate, dl-a-tocopherol polyethyleneglycol 1000 succinate, d-a-tocopherol polyethyleneglycol 1000 succinate or mixtures thereof.

7. The pharmaceutical composition of claim 4, wherein the fatty acid derivative is an ester with glycerol, propylene glycol, sorbitol, sucrose, glucose, polyethylene glycol, an alpha-hydroxy acid or mixtures thereof.

8. The pharmaceutical composition of claim 4, wherein the ester is a polyoxyl castor oil derivative, a PEG-8 caprylic/capric glyceride, a polysorbate, sorbitan monooleate, a medium chain mono-, di-, or triglyceride, a acetylated monoglyceride, a linoleoyl monoglyceride, a lauroyl macrogol-32glyceride or mixtures thereof.

9. The pharmaceutical composition of claim 1 or claim 3 wherein the release modulator is an osmotic pump, a slowly dissolving salt of complex, an erodible matrix, an exchange resin, a wax, an insoluble carrier, a polymeric matrix, a polymeric coating, a fatty acid, a fatty alcohol, a fatty acid derivative, a fatty alcohol derivative or a tocol derivative.

10. The pharmaceutical composition of claim 9 wherein the release modulator is a polymeric matrix, a polymeric coating, a wax, a fatty alcohol, a fatty acid, a fatty alcohol derivative, or a fatty acid derivative, a tocol derivative or mixtures thereof.

11. The pharmaceutical composition of claim 10 wherein the polymeric matrix or polymeric coating is a cellulose derivative, an acrylic polymer, a polyvinylpyrrolidone copolymer, shellac, polyvinyl acetate phthalate, a high molecular weight polysaccharide gum or mixtures thereof.

12. The pharmaceutical composition of claim 9 wherein the tocol derivative is a-tocopherol, a-tocopherol acetate, a-tocopherol nicotinoate, a tocopherol succinate, a-tocopherol polyethyleneglycol succinate, a-tocopherol polyethylene glycol 400 succinate, or mixtures thereof.

13. The pharmaceutical composition of claim 9 wherein the release modulator is microcrystalline wax, hydrogenated vegetable oil, glycerol dibehenate, glycerol distearate, glycerol dipalmitate, glycerol palmitostearate, a lauroyl macrogol 32 glyceride, a stearoyl rnacrogol-32 glyceride, calcium steroyl lactylate, stearic acid, stearoyl alcohol, sucrose distearate, sucrose palmitate, sucrose dipalmitate, yellow wax, white wax, carnauba wax, nonionic emulsifying wax, cetyl ester wax or mixtures thereof.

14. The pharmaceutical composition of claim 1, wherein the aqueous solubility of the drug is less than about 100 pg/ml.

15. The pharmaceutical composition of claim 1, wherein the aqueous solubility of the drug is less than about 50 pg/ml.

16. The pharmaceutical composition of claim 1, wherein the aqueous solubility of the drug is less than about 25 pg/ml. 20

17. The pharmaceutical composition of claim 1, wherein the release is over an extended period of time.

18. The pharmaceutical composition of claim 17, wherein the period of 25 time is more than about 1 hour.

19. The pharmaceutical composition of claim 17, wherein the period of time is more than about 2 hours.

20. The pharmaceutical composition of claim 17, wherein the period of time is between about 2 hours and about 24 hours.

21. The pharmaceutical composition of claim 1, wherein the solubilizer increases the solubility of the drug by at least 25% in comparison to the intrinsic aqueous solubility of the drug.

22. The pharmaceutical composition of claim 1, wherein the release of the drug and solubilizer are synchronized with a correlation coefficient of greater than 0.80.

23. The pharmaceutical composition of claim 1, wherein the release of the 10 drug and solubilizer are synchronized with a correlation coefficient of greater than 0.90.

24. The pharmaceutical composition of claim 1, wherein the release of the drug and solubilizer are synchronized with a correlation coefficient of greater than 0.95.

25. The pharmaceutical composition of claim 1 including one or more additives.

26. The pharmaceutical composition of claim 1, wherein the solubilizer is d-a-tocopherol polyethylene glycol 1000 succinate or polyoxyl 40 hydrogenated castor oil and the release modulator is a-tocopherol succinate, glycerol dibehenate or hydroxypropylmethylcellulose.

27. The pharmaceutical composition of claim 26, including one or more additives.

28. The pharmaceutical composition of claim 27, wherein the solubilizer is d-a-tocopherol polyethylene glycol 1000 succinate, the release modulator is a30 tocopherol succinate and the additive is polyethylene glycol.

29. The pharmaceutical composition of claim 27, wherein the solubilizer is polyoxyl 40 hydrogenated castor oil and the release modulator is hydroxypropylmethylcellulose.

30. The pharmaceutical composition of claim 1, wherein the aqueous solubility of the drug is dependent on pH.

31. The pharmaceutical composition of claim 30, wherein the drug has a pKa of less than or equal to about 9.0.

32. The pharmaceutical composition of claim 30, wherein the drug is carvedilol, amiodoarone, dronederone, risperdone or ziprasidone.

33. A oral dosage form comprising:
a therapeutically effective amount of a drug;
a solubilizer; and
a release modulator;
wherein the release of the drug and solubilizer are synchronized.

34. A solid oral dosage form comprising:
a therapeutically effective amount of a drug;
a solubilizer; and 30 a release modulator;
wherein the release of the drug and solubilizer are synchronized.

35. The pharmaceutical composition of claim 1, wherein the drug is testosterone undecanoate.

36. The pharmaceutical composition of claim 1, wherein the drug is megestrol acetate.

37. The pharmaceutical composition of claim 1, wherein the drug is clonazepam

38. The pharmaceutical composition of claim 1, wherein the drug is anagrelide.

39. The pharmaceutical composition of claim 1, wherein the drug is acamprosate.

40. The pharmaceutical composition of claim 1, wherein the drug is selected from the comprising valsartan, telmisartan, candesartan cilexetil, olmesartan medoxomil, and irbesartan.
